SEQUENCE LISTING (21 sequences)

SEQ ID NO: 1 - DNA, 12 bp, Artificial Sequence
  Synthetic
tcatctagtgac

SEQ ID NO: 2 - DNA, 12 bp, Artificial Sequence
  Synthetic
tcatctagtaac

SEQ ID NO: 3 - DNA, 24 bp, Artificial Sequence
  Synthetic
tcatctagtgantcatctagtgac

SEQ ID NO: 4 - DNA, 36 bp, Artificial Sequence
  Synthetic
tcatctagtgantcatctagtgantcatctagtgac

SEQ ID NO: 5 - DNA, 48 bp, Artificial Sequence
  Synthetic
tcatctagtgantcatctagtgantcatctagtgantcatctagtgac

SEQ ID NO: 6 - DNA, 24 bp, Artificial Sequence
  Synthetic
gtcactagatgngtcactagatga

SEQ ID NO: 7 - DNA, 10 bp, Artificial Sequence
  Synthetic
tcactagatg

SEQ ID NO: 8 - DNA, 12 bp, Artificial Sequence
  Synthetic
agtagatcactg

SEQ ID NO: 9 - DNA, 12 bp, Artificial Sequence
  Synthetic
cagtgatctact

SEQ ID NO: 10 - DNA, 12 bp, Artificial Sequence
  Synthetic
agtagatcattg

SEQ ID NO: 11 - DNA, 12 bp, Artificial Sequence
  Synthetic
gtcacnagatga

SEQ ID NO: 12 - DNA, 12 bp, Artificial Sequence
  Synthetic
gncactaganga

SEQ ID NO: 13 - DNA, 12 bp, Artificial Sequence
  Synthetic
gncacnaganga

SEQ ID NO: 14 - DNA, 12 bp, Artificial Sequence
  Synthetic
ngnnactagtga

SEQ ID NO: 15 - DNA, 12 bp, Artificial Sequence
  Synthetic
gtcacnaganga

SEQ ID NO: 16 - DNA, 14 bp, Artificial Sequence
  Synthetic
ngtcactagatgan

SEQ ID NO: 17 - DNA, 14 bp, Artificial Sequence
  Synthetic
atcatctagtgaca

SEQ ID NO: 18 - DNA, 12 bp, Artificial Sequence
  Synthetic
ctactcagtaac

SEQ ID NO: 19 - DNA, 24 bp, Artificial Sequence
  Synthetic
tcatctagtgactcatctagcgac

SEQ ID NO: 20 - DNA, 36 bp, Artificial Sequence
  Synthetic
tcatctagtgactcatctagtgactcatctagtgac

SEQ ID NO: 21 - DNA, 48 bp, Artificial Sequence
  Synthetic
agtgatctacttcatctagtgacntcatctagtgactcatctagtgac

(19) United States
(12) Patent Application Publication
(10) Pub. No.: US 2017/0002355 A1
(43) Pub. Date: Jan. 5, 2017

(54) cGAP-PNA MULTIVALENT PEPTIDE NUCLEIC ACID LIGAND DISPLAY

(71) Applicant: THE USA, AS REPRESENTED BY THE SECRETARY, DEPART- MENT OF HEALTH AND HUMAN SERVICES, Bethesda, MD (US)

(72) Inventors: Daniel H. Appella, Rockville, MD (US); Andrew V. Dix, Eugene, OR (US); Ethan Englund, Germantown, MD (US); Kara George Rosenker, Rockville, MD (US)

(73) Assignee: THE USA, AS REPRESENTED BY THE SECRETARY, DEPART- MENT OF HEALTH AND HUMAN SERVICES, Bethesda, MD (US)

(21) Appl. No.: 15/112,981

(22) PCT Filed: Jan. 16, 2015
(86) PCT No.: PCT/US2015/011730

§ 371 Date: Jul. 20, 2016

Related U.S. Application Data
(60) Provisional application No. 61/929,893, filed on Jan. 21, 2014.

(51) Int. Cl. C12N 15/117; A61K 39/00; A61K 51/04; A61K 47/48

(52) U.S. Cl. CPC C12N 15/117; A61K 47/48092; A61K 39/00; A61K 51/0497; A61K 2039/6025; C12N 2310/52; C12N 2310/151; C12N 2310/3513; C12N 2310/3181

(57) ABSTRACT

Described herein are compositions composed of peptide nucleic acid strands. In some aspects the peptide nucleic acid strands are complementary to at least a portion of another peptide nucleic acid strand that may have one or more gamma substituents, where the ratio of PNA strands is least 1:1. Certain gamma substituents are capable of effecting attachment of a PNA strand to a cell. The disclosure also concerns construction of nanostructure platforms and vaccines and use of the inventive compositions in inhibiting disease states in mammals.

### CROSS-REFERENCE TO A RELATED APPLICATION

This patent application claims the benefit of U.S. Provisional Patent Application No. 61/929,893, filed Jan. 21, 2014, which is incorporated by reference.

### BACKGROUND OF THE INVENTION

Multivalent (or polyvalent) interactions refer to the simultaneous binding of multiple ligands on the surface of one molecular entity to multiple receptors on another. The strength and specificity of multivalent interactions depends on the cumulative effect of all the ligands and all the receptors involved in the process. Within a multivalent array, a single, isolated ligand-receptor interaction may actually be weak; however, the combined effect of multiple ligand-receptor interactions can be very strong. Such multivalent interactions occur throughout biology, and are important in numerous processes, such as those involving receptors at the surfaces of cells. For example, cell attachment, wound healing, and the immune response are basic examples where multivalent interactions are important. Therefore, multivalent interactions can be directly linked to cancer metastasis, blood clotting, and the generation of antibodies from a vaccination (see generally, Mammen et al., Angew. Chem. Int. Ed., 37:2754-94 (1998)).

Mimicking multivalent interactions on a synthetic scaffold is challenging, especially when large numbers of ligands (such as 5 or more) need to be displayed. There are numerous synthetic scaffolds that have been developed, but there are significant limitations that remain. Ideally, a scaffold for the multivalent display of ligands should be easily manipulated to display anywhere from 1 up to about 200 ligands in a controlled manner. Well-defined synthetic scaffolds have been developed for the display of small numbers of ligands. Such systems are good because a single synthetic entity can be made and isolated, but it is rare that such systems display more than 5 ligands. Beyond this, well-defined synthetic scaffolds become very challenging to make. To study the multivalent effects of larger numbers of ligands, scientists rely on synthetic systems that are less well-defined and consist of mixtures. In this area, it is common to use polymers, dendrimers, proteins, and synthetic nanostructures (such as gold nanoparticles) as the synthetic scaffold to support larger numbers of ligands. Unfortunately, these larger systems are heterogeneous mixtures where the number of ligands per scaffold cannot be rigorously defined. In these cases, scientists determine an average number of ligands per scaffold or report the range of ligands per scaffold. Heterogeneous mixtures are often not acceptable by FDA standards for application as a therapeutic. As such, improved scaffolds are needed.

### BRIEF SUMMARY OF THE INVENTION

Described herein is a different type of PNA that remains soluble at longer lengths. The new type of PNA is termed “cGAP-PNA,” The “c” stands for complementary because the cGAP-PNA has a nucleobase sequence that is complementary to the ligand-modified PNA (hereinafter “L-PNA”) sequence. The “GAP” is any chemical group that interrupts adjacent PNA nucleobase sequences which are complementary to the L-PNA. In most cases, the GAP between adjacent PNA sequences in a cGAP-PNA is an amino acid, such as N,N-dimethyl lysine; however, other amino acids may be used, as well as other suitable chemical groups described herein.

Described herein are macromolecules having a plurality of linked peptide nucleic acid (PNA) strands, where each of the PNA strands is independently composed of a plurality of nucleobase subunits, and each PNA strand is covalently linked to at least one other PNA strand via an amino acid linker (i.e., a “GAP”). In some embodiments a PNA strand will have between 2 and 50 nucleobases. In some embodiments the linked PNA strands may form a linear arrangement, such that they are linked successively in an end-to-end manner. In one such embodiment the linked PNA strands form an open-ended single linear arrangement (as might be representative of a straight line). In another embodiment, the linked PNA strands form an closed-ended linear arrangement (as might be representative of a circle). In some embodiments the linked PNA strands may be arranged in a branched arrangement. The length of a PNA strands may differ, even within a single arrangement. For example, PNA stands linked in an arrangement may differ in length, such that some strands are shorter than others. Conversely, in some embodiments, the described compositions may be made of PNA strands that are the same length.

The linker used to form the described PNA arrangements may be an amino acid composition. In one embodiment the linker may me a naturally occurring amino acid. In another embodiment the linker may be a synthetic amino acid. In yet another embodiment the amino acid compound may be any chemical compound that includes a terminal amino group and a terminal carboxyl group. The described linkers may mediate the linkage of two or more of the described PNA strands. In some embodiments, the linker will join only two PNA strands. However, in other embodiments, a single linker may join three, four, five, six, seven, or more PNA strands. The degree to which a single linker mediates the conjugation between PNA strands will usually depend on the desired structure of the resulting cGAP-PNA.

In some aspects, the invention concerns macromolecules having a linked PNA (GAP-PNA) bound to an L-PNA. In some embodiments each PNA strand can have from 2 to 50 nucleobase subunits; the L-PNA strand will have one or more gamma substituents; the PNA strands are complementary to at least a portion of one another, with the ratio of cGAP-PNA strands to L-PNA strands being at least 1:1. These macromolecules may form at least a partially double-stranded GAP-PNA-L-PNA macromolecule, termed a L-PNA:PNA(GAP). Certain embodiments may contain an L-PNA with a backbone having at least one cyclopentyl residue.

Some macromolecules of the invention have gamma substituents that are capable of binding to a receptor on the surface of a cell, binding to a cell surface molecule, or eliciting an immune response. In some embodiments, the ratio of cGAP-PNA strands to L-PNA strands is 2:1 to 10:1. In certain embodiments, the ratio is 3:1 to 7:1 or 4:1 to 6:1.

In some embodiments gamma substituents of the L-PNA, independently, include R—NX¹X², where: R is a C₁-C₁₂ alkyl; X¹ and X² are independently selected from H, biomolecules, fluorescent groups, metal ligands, Michael acceptors, azides, alkynes, and thiols; where at least one of X¹ and X² are other than H. In certain embodiments, X¹ and X² are independently selected from H, biotin, fluorescein, thiazole orange, acridine, pyrene, Alexafluor Dyes, polypeptide, sugars (such as mannose or lactose), nucleic acid derivatives, oligonucleotides, RGD (Arg-Gly-Asp) and cyclic RGD. Additional groups and ligands that may be attached to a PNA for multivalent display include, but are not limited to, cyclodextrins, porphyrins, polyhedral cage compounds containing boron, biotin, 1,4,7,10-tetraazacyclododecane-N,N′, N″,N″-tetraacetic acid (DOTA), diethylene triamine pentaacetic acid (DTPA), a cryptand, a crown ether (12-crown-4, 15-crown-5, 18-crown-6, dibenzo-18-crown-6, and diaza-18-crown-6, or derivatives, for example), a pyridine-containing ligand, or calixarenes (such as calix[4]arenes, e.g., cone-4-tert-butylcalix[4]arenetetra(diethylamide), and calix[6] arenes. In some embodiments, derivatives of the aforementioned X¹ and X² groups and ligands may be utilized. In addition to gamma substituents, the N-terminal PNA residues of the compounds described herein can also include substituents (R2 and R3) on the nitrogens of the N-termini. Accordingly, a single PNA residue can have up to 3 substituents, one gamma substituent and two terminal substituents.

In some embodiments the individual L-PNA residues described herein can have one substituent. In some instances this substituent will be conjugated to the gamma carbon of the L-PNA residue. In some embodiments this substituent will be conjugated to the terminal nitrogen of the L-PNA residue. An L-PNA residue of this nature will be an individual residue in some embodiments. However, it may also be one of multiple PNAs in a larger strand. In some embodiments the L-PNAs described herein can be complexed with cGAP-PNAs to form a L-PNA:PNA(GAP).

In some embodiments the individual L-PNA residues described herein can have two substituents. In some instances at least one of the two substituents will be conjugated to the gamma carbon of the L-PNA residue. In some embodiments, at least one of the two substituents will be conjugated to a terminal nitrogen residue. In some instances at least one of the two substituents will be conjugated to the gamma carbon of the L-PNA residue and the other will be conjugated to a terminal nitrogen residue. An L-PNA residue of this nature will be an individual residue in some embodiments. However, it may also be one of multiple PNAs in a larger strand. In some embodiments the L-PNAs described herein can be complexed with cGAP-PNAs to form a L-PNA:PNA(GAP).

In some embodiments the individual L-PNA residues described herein can have three substituents. In some instances at least one of the substituents will be conjugated to the gamma carbon of the L-PNA residue. In some embodiments, two substituents will be conjugated to a terminal nitrogen residue. In some instances at least one of the substituents will be conjugated to the gamma carbon of the L-PNA residue and the other two will be conjugated to a terminal nitrogen residue. An L-PNA residue of this nature will be an individual residue in some embodiments. However, it may also be one of multiple PNAs in a larger strand. In some embodiments the L-PNAs described herein can be complexed with cGAP-PNAs to form a L-PNA:PNA(GAP).

The invention also concerns methods of treating or inhibiting a disease state in a mammal comprising administering to said mammal an effective amount of a macromolecule described herein wherein at least some of the gamma substituents are selected to bind to a receptor on the surface of a cell associated with said disease state, to hinder the ability of a cell surface molecule to interact with a ligand that may trigger or prolong a disease state, or elicit an immune response. In some embodiments, the disease state is related to, independently, cancer; infectious diseases caused by HIV, influenza, rhinovirus, rotavirus, E. coli, anthrax or cholera; diabetes (type 2), Chagas disease, chronic inflammatory diseases, and autoimmune diseases (see generally, Hecht et al., Curr. Opin. in Chem. Biol., 13:354-59 (2009) and Mammen et al.).

In yet another aspect, the invention concerns methods of forming nanostructure platforms by contacting a cGAP-PNA strand with an L-PNA strand, wherein the L-PNA strand has:

(i) from 2 to 50 nucleobase subunits, and

(ii) one or more gamma substituents;

wherein the ratio of the L-PNA strands to the cGAP-PNA strand is greater than 1:1 and said PNA strands are complementary to a portion of one another.

### BRIEF DESCRIPTION OF THE SEVERAL VIEWS OF THE DRAWING(S)

FIG. 1A depicts a representation of a L-PNA:DNA duplex as a chemical structure with the γ-lysine side chain modification highlighted in red. XAC is connected to the side chain by two mini-PEG (8-amino-3,6-dioxaoctanoic acid) linkers. FIGS. 1B-1D depict representations of a L-PNA 12-base oligomer bound to complementary DNA with one XAC ligand (FIG. 1B), two XAC ligands (FIG. 1C), and three XAC ligands per L-PNA (FIG. 1D).

FIG. 2A depicts a L-PNA:DNA multivalent library with the associated IC₅₀ and β values for binding to A2A adenosine receptor (“AR”). FIG. 2BA depicts a multivalent landscape highlighting the relationships between the A, B, and C type L-PNA constructs when annealed to various lengths of DNA.

FIG. 3A depicts a representation of a bivalent L-PNA:PNA duplex as a chemical structure. The L-PNA contains two adjacent ligand-bearing sidechains with a spacing of one base pair (bp), which is approximately 3.7 Å. FIG. 3B shows several bivalent L-PNA:PNAs that were generated to determine the effects of axial spacing on receptor binding ability. Along with the monovalent A1_{P} control, the four bivalent complexes are summarized including their IC₅₀ values, the change in axial distance between the ligand-sidechains, and the value of the complex compared to A1_{P}.

FIG. 4 depicts a statistical model. The model assumes that only a discrete number of different binding states exist between L-PNA:PNA and the receptor. A subset of these states are highlighted for the monovalent complex (FIG. 4A) and the bivalent complexes (FIG. 4B). Using every possible ligand configuration of A1_{P} and B1_{P} complexes, the model samples an ensemble of states in accordance with a specific fraction of protein in the dimeric state, with the results shown in FIG. 4C. This information can then be extrapolated and plotted as the fraction of receptors in the dimeric state (D) versus the error (ε) between the theoretical and experimentally observed data as shown in FIG. 4D. A region of minimal error is designed as the “ideal region.”

FIGS. 5A-5D depicts a molecular model of a proposed A_{2A} dimer that was built based on a known antagonist-bound crystal structure of the monomer. The B_{(6,10)}1_{P} complex was modeled with the dimer, both (FIG. 5A) with and without (FIG. 5B) the phospholipid bilayer (cellular membrane). Using a subset of the data from the statistical model, possible side chain organizations are superimposed on the model for the B_{(2,3)}1_{P} (FIG. 5C) and B_{(6,10)}1_{P} (FIG. 5D) complexes.

FIGS. 6A and 6B show a L-PNA:PNA multivalent landscape. L-PNA:PNA multivalent library with the associated IC₅₀ and β values are shown in FIG. 6A. Complex B_{(6,10)4P} was also screened against the A_{2A}AR homologues A₁AR (260 nM) and A₃AR (180 nM). Multivalent landscape highlighting the relationships between the A (red), B_{(2,10)}, B_{(2,10)}, B_{(6,10)} and C type L-PNA constructs when annealed to various lengths of complementary PNA are shown in FIG. 6B.

FIG. 7 shows a structural representation of PNAa.

FIG. 8 shows a structural representation of L-PNA A.

FIG. 9 shows a structural representation of PNAb.

FIG. 10 shows a structural representation of PNA B.

FIG. 11 shows a structural representation of PNAc.

FIG. 12 shows a structural representation of PNA C.

FIG. 13 shows a structural representation of PNA b_{2,3}.

FIG. 14 shows a structural representation of L-PNA B_{2,3}.

FIG. 15 shows a structural representation of PNA b_{6,10}.

FIG. 16 shows a structural representation of L-PNA B_{6,10}.

FIG. 17 shows a structural representation of PNA b_{1,14}.

FIG. 18 shows a structural representation of L-PNA B_{1,14}.

FIG. 19 shows a structural representation of complement PNA1, having the structure Me₂Lys-TCA-TCT-AGT-GAC-Ac.

FIG. 20 shows a structural representation of complement PNA1₀,₁₄), having the structure Me₂Lys-A-TCA-TCT-AGT-GAC-A-Ac.

FIG. 21 shows a structural representation of complement PNA1(2,3), having the structure Me₂Lys-TCA-TCT-AGT-AAC-Ac.

FIG. 22 shows a structural representation of complement PNA2, having the structure Me₂Lys-TCA-TCT-AGT-GAC-Me₂Lys-TCA-TCT-AGT-GAC-Me₂Lys-Ac.

FIG. 23 shows a structural representation of complement PNAm3, having the structure Me₂Lys-TCA-TCT-AGT-GAC-Me₂Lys-TCA-TCT-AGT-GAC-Me₂Lys-TCA-TCT-AGT-GAC-Me₂Lys-Ac.

FIG. 24 shows a structural representation of complement PNA4, having the structure Me₂Lys-TCA-TCT-AGT-GAC-Me₂Lys-TCA-TCT-AGT-GAC-Me₂Lys-TCA-TCT-AGT-GAC-Me₂Lys-TCA-TCT-AGT-GAC-Me₂Lys-Ac.

FIG. 25A shows a chemical structure of L-PNA:PNA duplex containing the ^{L}Kγ-PNA sidechain.

FIG. 25B shows chemical structures of D2R agonist (±)-PPHT and modified lysine residue X.

FIG. 25C shows L-PNA oligomer bound to complementary PNA with one (±)-PPHT ligand (A-type), two (±)-PPHT ligands (B-type), and three (±)-PPHT ligands (C-type) per PNA.

FIG. 25D shows that each L-PNA sequence is identified by its constituent parts; for example, an A2 complex contains 2 A-type L-PNA units annealed along a 24-residue cPNA.

FIG. 26 show a multivalent landscape highlighting the relationships between the A, B, and C type L-PHA constructs when annealed to various lengths of DNA.

### DETAILED DESCRIPTION OF THE INVENTION

Peptide nucleic acids (PNAs) are DNA mimics that maintain traditional Watson-Crick base pairing. The main difference between PNA and DNA is the backbone: DNA nucleotides are linked by negatively charged phosphodiester groups between adjacent riboses whereas PNA units are connected via charge-neutral amide bonds. PNA binds strongly and with sequence selectivity to complementary DNA, RNA, and even PNA to form double helical structures. In particular, PNA-PNA duplexes are extraordinarily stable to denaturation compared to similar duplexes composed of DNA or RNA.

Short PNA segments having γ-sidechains may be modified to display biologically relevant ligands. A PNA displaying one or more ligands (L-PNAs) can be annealed to a complementary DNA sequence that may support anywhere from 1 to 15 L-PNAs. These L-PNA:DNA complexes serve as a scaffold to organize the multivalent display of ligands that project from the L-PNA. Using this approach, a library of L-PNA:DNA complexes can be generated and each tested to find the optimal multivalent display for a specific biological effect.

There are some drawbacks to the L-PNA-DNA system, however. For example, the negative charge on the DNA backbone from the phosphodiester groups can lead to nonspecific binding. In addition, the DNA may be susceptible to enzymatic degradation. Furthermore, using known techniques PNAs can only be made up to about 20 nucleobases in length before they become highly insoluble.

As used herein, the term “reporter molecule” is to be understood to mean any group which is detectable by analytical means in vitro and/or in vivo and which confers this property to the conjugate. Some reporter molecules are a fluorescent molecule having fluorescence properties which are a function of the concentration of the molecule. Other reporter molecules have an absorbance spectra that can be monitored for detection. Numerous reporter molecules are known to those skilled in the art and are suitable for use with the present invention. One preferred reporter molecule is biotin.

As used herein, the terms “a”, “an”, “the” and the like refer to both the singular and plural unless the context clearly indicates otherwise.

The term “amino acid” is to be construed broadly, in a chemical sense rather than a biological sense. Accordingly, the term denotes any chemical group having a carboxyl terminus and an amino terminus. While naturally occurring and synthetic biological amino acid compositions fall within the scope of the term, the definition is not so limited as to only include these chemical compositions.

The term “cross reactive groups” refers to at least two groups that are capable of reacting to form a covalent bond linking the first and second PNAs.

A “GAP” is any chemical group that interrupts adjacent PNA nucleobase sequences which are complementary to an L-PNA. Commonly, though not always, GAPs are amino acids.

The term “L-PNA” is used to represent a PNA base having a ligand attached to it; however, the term should not be considered limited to denote a PNA bound to a moiety known to be a physiological ligand. The term “L-PNA” can denote a PNA linked to other biomolecules as well, such as a receptor, antigenic molecule, viral or bacterial coat protein, or protein fragments thereof.

The technology presented herein overcomes the barriers that persist for the current multivalent scaffolds. The technology uses peptide nucleic acids (PNAs) which are backbone substituted to provide a multivalent scaffold (L-PNA:PNA(GAP)). Preferably in the L-PNA:PNA(GAP), one or more sidechains have been introduced at one or more gamma carbons of the L-PNA backbone.

PNAs are synthetic molecules that possess the bases derived from DNA. Similar to DNA, the sequence of bases on a PNA determines the complementary sequence of nucleic acids to which a PNA will bind. Sidechains at the gamma carbon of PNA may have a nitrogen atom to facilitate attachment of ligands to the sidechains that extend off of the backbone.

As used herein, the term “macromolecule” refers to a plurality of linked peptide nucleic acid strands.

The L-PNAs described herein can interact with the complementary nucleobases of a cGAP-PNA to form a L-PNA:PNA(GAP) complex that is at least partially double stranded. In the instance where the entirety of the L-PNA is complementary to the entirety of the cGAP-PNA the entire complex will be have double-stranded nucleobase segments. The cGAP-PNA composition could be described as a composition with a plurality of linked peptide nucleic acid (PNA) strands, wherein each of said strands is independently composed of a plurality of nucleobase subunits, and each PNA strand is covalently linked to at least one other PNA strand via an amino acid linker. In some aspects the ratio of L-PNA to cGAP-PNA is greater than 1:1. An example of a cGAP-PNA is shown below, where the Me2Lys “GAP” is used to link two PNAs that are complementary to L-PNAs (not shown):

GTC-ACT-AGA-TGA-Me2Lys-GTC-ACT-AGA-TGA

(complement to L-PNA) (GAP) (complement to L-PNA).

As an example, a cGAP-PNA may be 60 nucleobases long and support assembly of 5 complementary L-PNAs (each with 12 nucleobases) that bear specific y ligands. In such an embodiment the cGAP-PNA has 4 GAPs within the sequence, and each GAP is a hydrophilic amino acid, such as N,N-dimethyl lysine amino acid. The presence of the GAPs helps in the synthesis and aqueous solubility of the final molecule. With this improvement, the lysine GAP overcomes the hydrophobicity and poor water solubility drawbacks in earlier PNA technology and expands the potential to synthesize PNAs of very long length.

The described cGAP-PNA strands are composed of at least one PNA strand covalently linked to at least one other PNA strand via an amino acid linker (i.e., a “GAP”). In some embodiments a PNA strand will have between 2 and 50 nucleobases. In some embodiments the linked PNA strands may form a linear arrangement, such that they are linked successively in an end-to-end manner. In one such embodiment the linked PNA strands form an open-ended single linear arrangement (as might be representative of a straight line). In another embodiment, the linked PNA strands form a closed-ended linear arrangement (as might be representative of a circle). In some embodiments the linked PNA strands may be arranged in a branched arrangement. The length of the PNA strands may differ, even within a single arrangement. For example, PNA stands linked in an arrangement may differ in length, such that some strands are shorter than others. Conversely, in some embodiments, the described macromolecules may be made of PNA strands that are the same length. Furthermore, the described GAP-PNA strands may be designed to include segments of nucleobases that are complementary to another PNA strand, such as an L-PNA. In some embodiments the entirety of the GAP-PNA will be complementary to an L-PNA. In some embodiments only a portion of the GAP-PNA will be complementary to an L-PNA. For example, a cGAP-PNA can be made of repeating 15 nucleobase segments each having two 5 base sub-segments complementary to the same L-PNA sequence, where the 5 base sub-segments are separated by 5 bases that are not complementary to the L-PNA. This would allow for spacing of the annealed L-PNA strands on the resulting L-PNA:PNA(GAP).

The described L-PNA:PNA(GAP)s allow for the assembly of multiple L-PNAs on a single molecule. Assembly in this manner allows the cGAP-PNAs to acts as the template to assemble multiple L-PNAs. For example, an L-PNA that consists of 12 bases and has one ligand attached to a sidechain, can display 10 of these ligands in a multivalent array by complexing these L-PNAs to a 120-base cGAP-PNA sequence that has the appropriate complementary sequence to the L-PNAs. With this system, a library of different entities with different numbers of ligands can readily be produced. For instance, switching from displaying 10 ligands to 5 is easily accomplished by simply using a different (shorter) L-PNA and the same cGAP-PNA. With this system, one is not restricted to just one ligand per L-PNA. The chemistry of the present invention allows attachment of multiple sidechains to the original L-PNA sequence so that, if desired, a sidechain can be attached at every position in the final L-PNA. In this case, a 12 base L-PNA with a ligand-bearing sidechain at every position in the backbone could be used to display 120 ligands if assembled onto a 120-base cGAP-PNA sequence that has the complementary sequence repeated 10 times. The versatility and accuracy of the system of the present invention is unparalleled by current multivalent scaffolds.

The cGAP-PNAs described herein make use of linker compounds (“GAPs”) to join PNA segments. The linkers, or GAPs, are commonly an amino acid compound, having a terminal amino group and a terminal carboxyl group. In some embodiments the linker is a naturally occurring, biological, amino acid. In some embodiments the linker is a synthetically produced, biological, amino acid. In some embodiments the linker is a chemical compound that is neither a naturally occurring or synthetic biological amino acid, but nonetheless has a terminal amino group and a terminal carboxyl group. In some embodiments the linker is N,N-dimethyl-lysine. In some embodiments the linker is A T,N-dimethyl-L-lysine. In some embodiments a linker may have more than one amino group and more than one carboxyl group, such that it may mediate linking more than 2 PNAs. In some embodiments a single linker may mediate the conjugation of 3 PNAs. In some embodiments a single linker may mediate the conjugation of 4 PNAs. In some embodiments a single linker may mediate the conjugation of 5 PNAs. In some embodiments a single linker may mediate the conjugation of 6 PNAs. In some embodiments a single linker may mediate the conjugation of 7 PNAs. In some embodiments a single linker may mediate the conjugation of 8 PNAs. In some embodiments a single linker may mediate the conjugation of 9 PNAs. In some embodiments a single linker may mediate the conjugation of 10 or more PNAs. Having linkers with the ability to join 3 or more PNAs allows for the ability to form branched cGAP-PNA structures, which can then be used as described herein to form branched L-PNA:PNA(GAP) scaffolds.

Also described herein are L-PNA segments that are themselves linked by GAPs. Such embodiments allow for the formation of elongated single-stranded L-PNA(GAP)s, similar to the GAP-PNAs described herein. Additionally, one could also produce complementary L-PNA(GAP)s that could then anneal with L-PNAs to form L-PNA:L-PNA(GAP) complexes. In some embodiments the ligands present on the L-PNA segments of these complexes may be the same. Alternatively, the ligands present on the L-PNA segments of the described L-PNA(GAP)s may differ. Furthermore, in one embodiment the L-PNA(GAP) strand may have a first ligand while the corresponding L-PNA has a second ligand, where the first and second ligands differ. Those of skill in the art will appreciate that a variety of ligand combinations are possible for the described L-PNA:L-PNA(GAP) complexes.

The described L-PNA:PNA(GAP)s are composed of L-PNAs bound to complementary GAP-PNAs. The number of L-PNA present on an individual cGAP-PNA segment can vary depending on a variety of factors, such as the length of the cGAP-PNA or the length of the corresponding L-PNA(s). Nonetheless each L-PNA:PNA(GAP) will have a ratio of L-PNA segments for to each individual cGAP-PNA segment. It should be understood, however, that this ratio can vary within the same L-PNA:PNA(GAP), since the individual cGAP-PNA segments can anneal with different numbers of L-PNAs depending on the complementarity of the PNAs for one another. In some embodiments the ratio of L-PNA to an individual cGAP-PNA is greater than 1:1. In some embodiments the ratio of L-PNA to an individual cGAP-PNA is 1:1. In some embodiments the ratio of L-PNA to an individual cGAP-PNA is 2:1. In some embodiments the ratio of L-PNA to an individual cGAP-PNA is 3:1. In some embodiments the ratio of L-PNA to an individual cGAP-PNA is 4:1. In some embodiments the ratio of L-PNA to an individual cGAP-PNA is 5:1. In some embodiments the ratio of L-PNA to an individual cGAP-PNA is 6:1. In some embodiments the ratio of L-PNA to an individual cGAP-PNA is 7:1. In some embodiments the ratio of L-PNA to an individual cGAP-PNA is 8:1. In some embodiments the ratio of L-PNA to an individual cGAP-PNA is 9:1. In some embodiments the ratio of L-PNA to an individual cGAP-PNA is 10:1. In some embodiments the ratio of L-PNA to an individual cGAP-PNA is 11:1. In some embodiments the ratio of L-PNA to an individual cGAP-PNA is 12:1. In some embodiments the ratio of L-PNA to an individual cGAP-PNA is 13:1. In some embodiments the ratio of L-PNA to an individual cGAP-PNA is 14:1. In some embodiments the ratio of L-PNA to an individual cGAP-PNA is 15:1. In some embodiments the ratio of L-PNA to an individual cGAP-PNA is 16:1. In some embodiments the ratio of L-PNA to an individual cGAP-PNA is 17:1. In some embodiments the ratio of L-PNA to an individual cGAP-PNA is 18:1. In some embodiments the ratio of L-PNA to an individual cGAP-PNA is 19:1. In some embodiments the ratio of L-PNA to an individual cGAP-PNA is 20:1. In some embodiments the ratio of L-PNA to an individual cGAP-PNA is greater than 20:1.

The L-PNA:PNA(GAP)s described herein can be used to target a protein. In some instances targeted proteins will be cell surface proteins. For example, cell surface proteins that are targets can be transmembrane proteins, lipid-anchored proteins, or peripheral proteins. Accordingly, the targeted proteins can be a cellular receptor or cellular adhesion molecule. In some instances the cell surface protein is an integrin. In some embodiments, the integrin may be α₁β₁, α₂β₁, α₄β₁, α₄β₇, α₅β¹, α₆β₁, α_{L}β₂, αMβ₂, α_{11b}β₃, αᵥβ₃, α_{V}β₅, α_{V}β₆, or α₆β₄. In some embodiments described herein the disclosed L-PNA:PNA(GAP)s may be used to bind to, or disrupt the activity of integrin α₆β₄. In some embodiments the targeted protein may be a G-coupled surface protein, such as a receptor. In other embodiments the cell surface proteins are ion channel linked receptors. In some embodiments the targeted protein may be an enzyme-linked receptor protein. In some aspects the targeted protein is a cadherin, such as E-cadherin, N-cadherin, cadherin 12, or P-cadherin. Selectins may also be targeted by the L-PNA:PNA(GAP)s described herein. For example, E-selectin, P-selectin, or L-selectin may be targeted by the described L-PNA:PNA(GAP)s. In some aspects, inter-cellular adhesion molecule 1 (ICAM-1) is targeted by the L-PNA:PNA(GAP)s described herein. In other embodiments, sialic acid on the cell surface may be targeted. C-type lectins may also be targeted by the L-PNA:PNA(GAP)s described herein. For example, some C-type lectins that may be targeted include: lecticans, asialoglycoprotein and DC receptors, collectins, NK cell receptors, multi-CTLD endocytic receptors, and thrombomodulin to name only a few. In addition, toll-like receptors may be targeted by the described L-PNA:PNA(GAP)s. Some toll-like receptors that may be targeted include TLR 1, TLR 2, TLR 3, TLR 4, TLR 5, TLR 6, TLR 7, TLR 8, TLR 9, TLR 10, TLR 11, TLR 12, or TLR 13.

Another advantage of the system of the present invention is that one accurately knows the number of ligands displayed in each case because the interaction between the L-PNA and the cGAP-PNA is well-defined. Furthermore, one can easily change the distance between adjacent ligands on the scaffold by inserting sections of non-complementary sequences in between the L-PNA-binding portions of the cGAP-PNA. An additional feature of this system is that it allows display of different types of ligands in a controlled, spatially-addressable manner. For example, if 2 different ligands (L1 and L2) each need to be displayed 3 times but in different specific orders (for example L1-L1-L1-L2-L2-L2 vs. L1-L2-L1-L2-L1-L2) this can be accomplished with the present system. To do this, one would make two different L-PNA nucleobase sequences (L-PNA1 and L-PNA2) and then attach L1 to the sidechain of L-PNA1 and L2 to the sidechain of L-PNA2. Examining the different order of ligands can be accomplished by making the appropriate cGAP-PNA sequence to display the ligands in the desired order. This process can be extended to more ligands. For instance, to display 10 different substituents (L1, L2, L3, L4, L5, L6, L7, L8, L9, L10) one would design ten different L-PNAs, each with a unique polynucleobase sequence, and attach one ligand to each L-PNA. Each of the ten ligands could then be assembled onto a cGAP-PNA sequence in a spatially-addressable manner. The technology uniquely allows small molecular rearrangements of multiple substituents to be explored. For the example of 10 substituents, the technology allows one to make ligand arrangements such as: L1-L2-L3-L4-L5-L6-L7-L8-L9-L10 or, reverse the position of L1 and L2, to give L2-L 1-L3-L4-L5-L6-L7-L8-L9-L10. With the ability to make libraries of cGAP-PNAs, one can even explore all combinations of the 10 substituents. The exploration of substituent libraries in this manner could also be extended to microarray technology as a way to assemble the substituent library and subsequently screen for activity and sequence information.

Those skilled in the art will understand that as many different substituents can be used as there are PNAs available for conjugation. Accordingly, the use of “10” substituents to illustrate the point of positional flexibility provided by the described system should not be seen as limiting. For example, L-PNAs described herein can be made of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 total bases. In some embodiments all of the substituents on an L-PNA strand are the same. In other embodiments the substituents of the L-PNA strand differ. The degree to which substituents differ relative to one another can vary such that all of the substituents can be unique to the instance where about 5% are unique. Alternatively, about 10% of the substituents could be unique. In some embodiments about 15% of the substituents are unique. In some embodiments about 20% of the substituents are unique. In some embodiments about 25% of the substituents are unique. In some embodiments about 30% of the substituents are unique. In some embodiments about 35% of the substituents are unique. In some embodiments about 40% of the substituents are unique. In some embodiments about 45% of the substituents are unique. In some embodiments about 50% of the substituents are unique.

Illustrative PNA constructs that have 1-5 side chains are known in the art (see e.g., WO 2011/143323 A2, which is incorporated by reference herein). Various side chains having moieties such as RGD and sugar derivatives (mannose and lactose, for example) are exemplified in WO 2011/143323 A2. Furthermore, the structures exemplified in the literature (e.g., WO 2011/143323 A2) can be used to attach a substituent via the terminal nitrogen-containing functional group on the side chain. In some embodiments, an L-PNA structure is represented by the following formula.

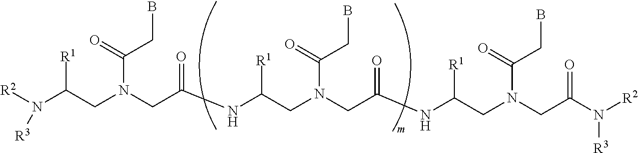

where R¹ is H or

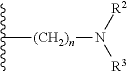

and m is 0 -48, n is an integer from 1-5. B is a natural (A, T, G, C) or non-natural nucleobase (such as J, isoguanine, or PPG).

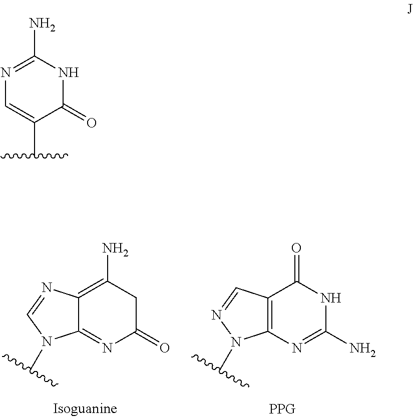 [SMILES: *c1cnc(N)[nH]c1=O ; *n1cnc2c1=NC(=O)CC=2N ; *n1ncc2c(=O)[nH]c(N)nc21]

As used herein, a “nucleobase subunit” is represented by a group of the structure:

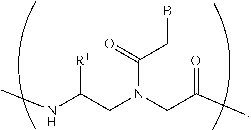

A PNA comprises a plurality of nucleobase subunits.

The range of groups for R² and R³ can vary from alkyl (such as CH₃ and derivatives such as substituted alkyls), to aryl (such as phenyl and obvious derivatives), to acyl (such as acetamido), to more complex linkers (such as PEG and associated variations) at the end of which are attached ligands for biomolecules, fluorescent groups, metal ligands, or other reactive groups (such as Michael acceptors, azides, alkynes, thiols) that may be used as a handle to attach other molecules. Some specific moieties that can be attached at the for R² and R³ positions include biotin, fluorescein, thiazole orange, acridine, pyrene, Alexafluor Dyes, polypeptides, sugars (such as lactose, mannose, or other oligosaccharides), nucleic acid derivatives (such as agonists or antagonists for adenosine receptors), oligonucleotides (such as G-quadruplexes). R² and R³ may also be peptide ligands such as RGD (Arg-Gly-Asp) and cyclic RGD.

Non-limiting examples of suitable ligands for biomolecules include GPCR agonists, GPCR antagonists, compounds that bind to integrin receptors, and compounds that bind to carbohydrate receptors. Non-limiting examples of suitable integrin receptor ligands can be found in, e.g., Vanderslice, P. et al., Expert Opin. Investig. Drugs, 2006, 15(1): 1235-1255 and Sun, C-C. et al., Anti-cancer Drugs, 2014, 25(1): 1107-1121, the disclosures of which are incorporated totally herein by reference. Non-limiting examples of GPCR agonists and antagonists can be found in, e.g., Insel, P. A. et al., Biochimica et Biophysica Acta, 2007, 1768: 994-1005, the disclosure of which is incorporated totally herein by reference. Non-limiting examples of suitable compounds that bind to carbohydrate receptors can be found in, e.g., Branson, T. R. et al., Chem. Soc. Rev., 2013, 42: 4613-4622, the disclosure of which is incorporated totally herein by reference. Non-limiting examples of suitable Michael acceptors include groups having a vinylcarbonyl, vinylcarboxyl, 1,2-dicarbonylethene, or 1,2-dicarbonylethyne moiety.

In addition to the examples listed above, any substituent that is useful in the desired biological interaction may be utilized in the present invention. Such moieties can be added to a PNA by standard chemical reactions and methods discussed herein. Examples of substituents which can be placed in the gamma position include primary amines, hydrophobic groups, polar groups, hydrophilic groups, aromatic groups, peptide ligands, receptor agonists, sugars, imaging agents, or mixtures thereof.

In making the constructs of the present invention, gamma-substituents can be introduced based on their utility in interacting with specific receptors or other biological interaction sites. In a particular PNA, more than one substituent may be utilized. In a particular L-PNA:PNA(GAP) scaffold, different L-PNAs may contain the same or different substituents depending on the target moiety.

In addition to the PNA depicted above, any of the numerous PNA variations that are known in the art can be utilized. Known PNA macromolecules include macromolecules represented by the following structures.

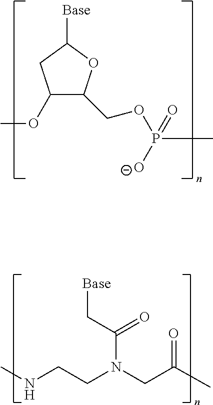

Natural and non-natural bases can be used in these structures and are well known by those skilled in the art.

Scheme 1 depicts a method of synthesis of a gamma-substituted monomer that can be used to make an L-PNA. The gamma substituent can serve as a point for further functionalization. These monomers can be converted to L-PNAs by methods known in the art. See, for example, Englund, E. A.; Appella, D. H. Angew. Chem. Int. Ed. Engl. 2007, 46, 1414.

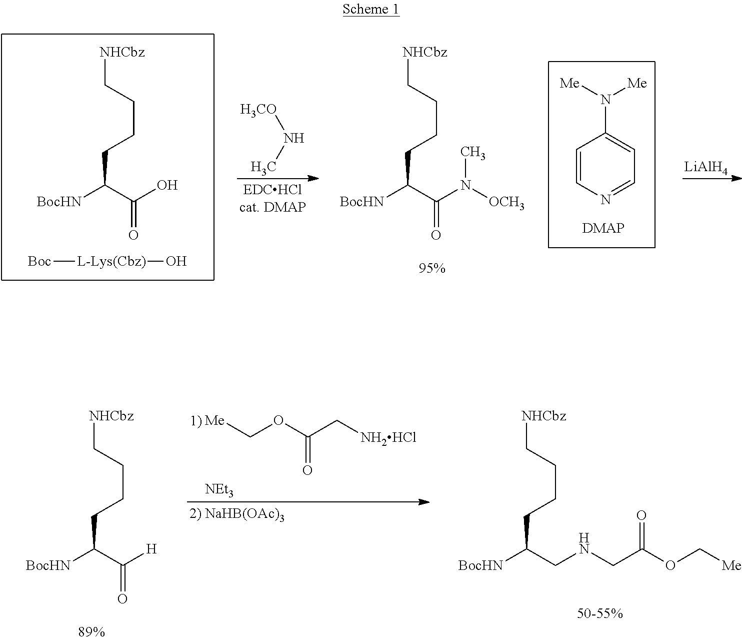

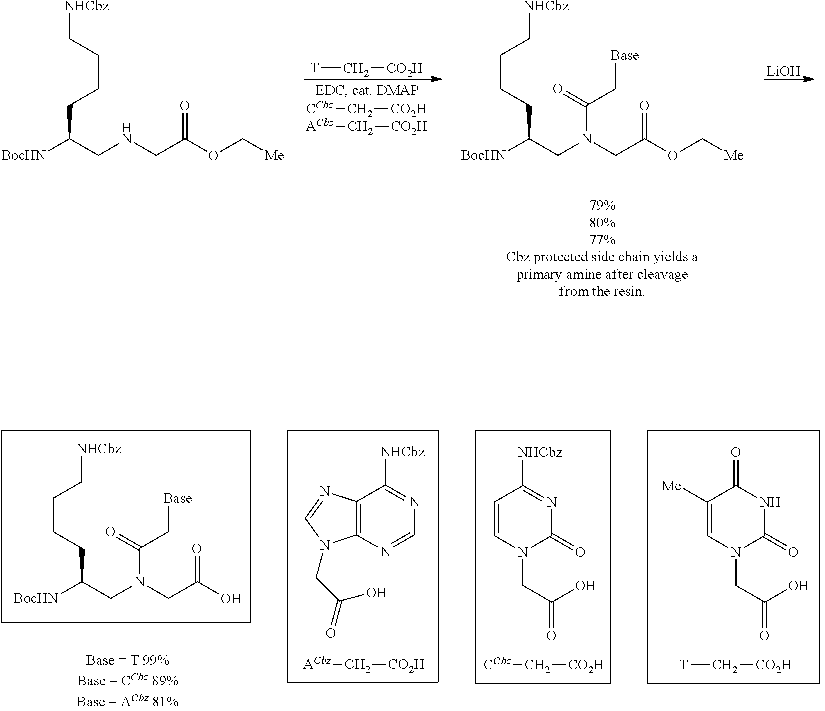

In scheme 2, use of FMoc allows for deprotection and coupling of amine while on resin. Reaction conditions can be adjusted to accommodate base labile Fmoc group. Typically, ester conversion to acid is done via hydrogenolysis rather than hydrolysis. See, for example, Englund, E. A.; Appella, D. H. Org. Lett. 2005, 7, 3465.

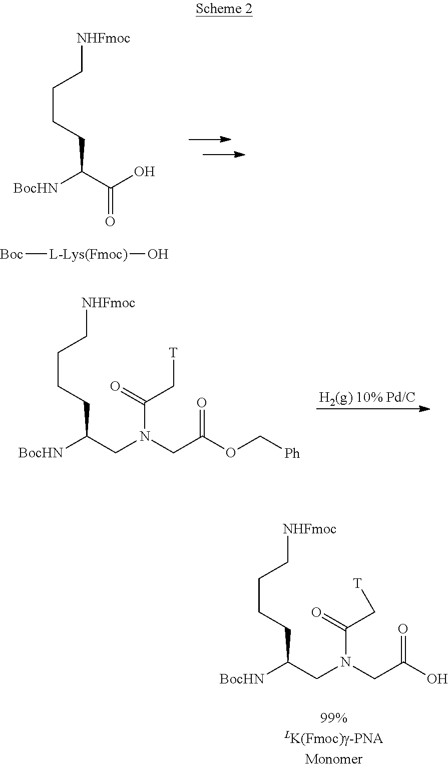

In Scheme 3, the oligomer is then cleaved from the resin (TfOH), purified via reverse phase HPLC, and characterized by mass spectrometry. See, for example, Koch, T.; et al. J. Peptide Res, 1997, 49, 80.

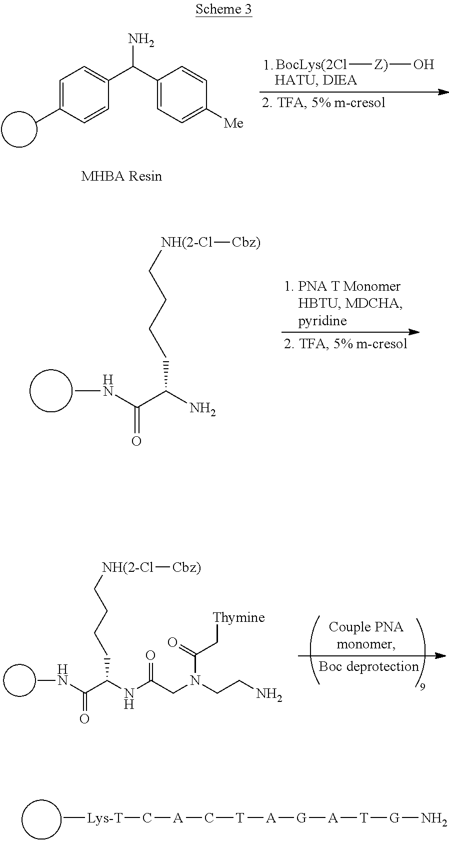

HBTU and HATU are defined by the structures below.

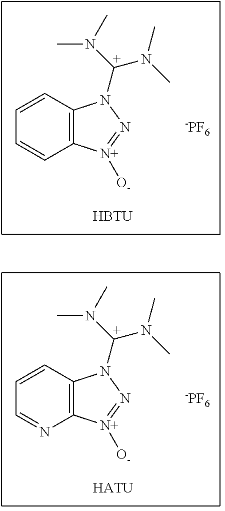

Sidechain modification of LK(Fmoc)γ-PNA is depicted in Scheme 4. This scheme provides access to wide variety of functionality. It is possible to conjugate some or all residues to different type of moiety via this process.

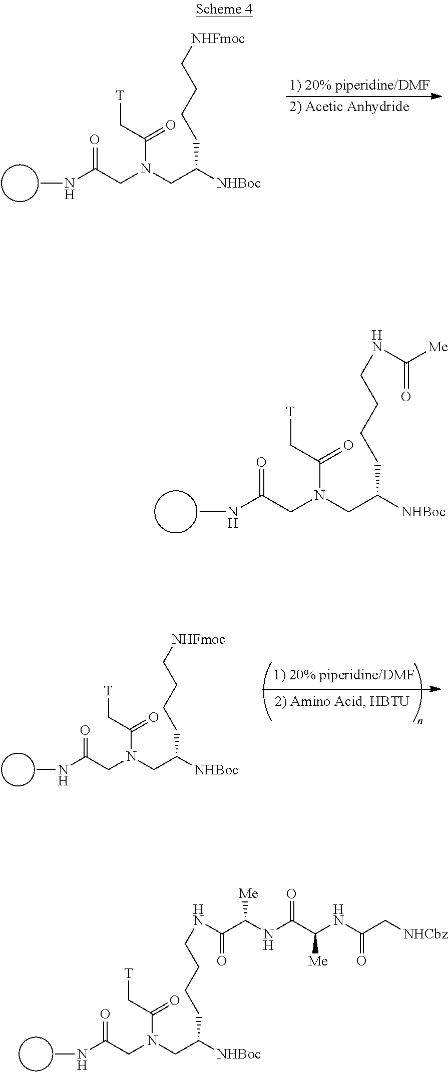

Adjacent PNAs that are assembled onto cGAP-PNAs can be cross-linked. Cross-linking reactive groups can be incorporated into these constructs by known techniques. In some embodiments, the cross-linking functional groups are attached in a terminal position in the L-PNA.

Cross-linking can be accomplished by use of cross-reactive functional groups. Many cross-reactive functional groups are known in the art and can be used with the present invention. In some embodiments, the cross-reactive functional groups can be of the formulas I and II. Reaction of a molecule of Group I with Group II produces a linkage shown by formula III.

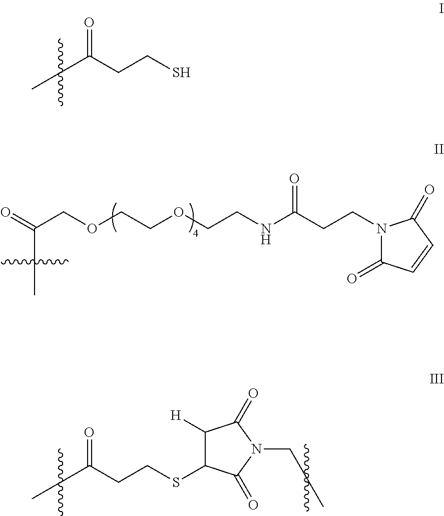

Some preferred PNAs contain trans-1,2-diaminocyclopentane which can potentially impact a broad range of scientific disciplines. Recent advances have improved the synthesis of trans-1,2-diaminocyclopentane. See, PCT Patent Application No. PCT/US2007/020466. These methods allow each nitrogen atom of cyclopentanediamine to be easily derivatized with identical or dissimilar groups. Incorporation of trans-1,2-diaminocyclopentane into PNAs has a beneficial effect on the recognition of GAP sequences. Methods for PNA and cyclopentane-modified PNA synthesis and acquisition of melting temperature data can be found in Pokorski, et al., J. Am. Chem. Soc. 2004, 126, 15067.

The L-PNA:PNA(GAP) scaffolds of the present invention can be utilized in pharmaceutical compositions. Such compositions can be produced by adding an effective amount of the scaffold composition to a suitable pharmaceutically acceptable diluent or carrier. Such carriers and diluents are well known to those skilled in the art.

The scaffolds and/or pharmaceutical compositions may be administered by methods well known to those skilled in the art. Such methods include local and systemic administration. In some embodiments, administration is topical. Such methods include ophthalmic administration and delivery to mucous membranes (including vaginal and rectal delivery), pulmonary (including inhalation of powders or aerosols; intratracheal, intranasal, epidermal and transdermal), oral or parenteral. Parenteral administration includes intravenous, intraarterial, subcutaneous, intraperitoneal or intramuscular injection or infusion; or intracranial (including intrathecal or intraventricular, administration).

Pharmaceutical compositions and formulations for topical administration include but are not limited to ointments, lotions, creams, transdermal patches, gels, drops, suppositories, sprays, liquids and powders. Utilization of conventional pharmaceutical carriers, oily bases, aqueous, powder, thickeners and the like may be used in the formulations.

The pharmaceutical compositions may also be administered in tablets, capsules, gel capsules, and the like.

Penetration enhancers may also be used in the instant pharmaceutical compositions. Such enhancers include surfactants, fatty acids, bile salts, chelating agents, and non-chelating non-surfactants. Such enhancers are generally described in U.S. Pat. No. 6,287,860, which is incorporated herein by reference.

The scaffolds described herein can be used to aid in treatment against diseases such as cancer (preventing metastasis, for example) and inhibition of HIV (by preventing attachment to the target cell's surface, for example). Other diseases include diabetes (Type 2), Chagas disease, chronic inflammatory diseases (such as celiac disease, vasculitis, lupus, chronic obstructive pulmonary disease, irritable bowel disease, atherosclerosis, arthritis, and psoriasis) and autoimmune diseases (such as diabetes mellitus type 1, Kawasaki disease, Graves' disease, Scleroderma).

In other applications, the scaffolds of the present invention can be utilized to generate vaccines. In one embodiment, a vaccine agent can be attached to a PNA to form a PNA analogous to an L-PNA, as described herein. The vaccine agent can be a moiety that resembles a disease-causing microorganism such as a weakened or killed forms of the microbe or its toxins. Attachment to the PNA can be made using standard chemical techniques at positions of the PNA discussed herein. For example, antigens from anthrax and cholera can be attached to the scaffolds, which can be combined with an adjuvant to stimulate antibody production. The compositions of the instant invention can be used in treating a disease state or can be used prophylactically.

Another example of a vaccine that can be produced from the inventive compositions would be functionally equivalent or similar to Prevnar™ (also known as Prevenar® in some countries). Prevnar™ is a vaccine produced by Wyeth and marketed by Pfizer which protects humans (typically administered at 2, 4, 5, and 12-15 months of age) from certain pneumococcal bacteria that can cause serious diseases such as meningitis and bacteremia.

Prevnar™ is a heptavalent vaccine, meaning it has seven different carbohydrates from seven different serotypes. The seven serotypes (strains) of S. pneumoniae included in the vaccine (4, 6B, 9V, 14, 18C, 19F, and 23F) were the strains that most commonly caused these serious diseases in children prior to the introduction of the vaccine. These carbohydrates, which can be derived from pneumococcus, are attached to a carrier protein to produce the vaccine. One application of the instant technology would be replace the carrier protein of the vaccine with a L-PNA:PNA(GAP) of the present invention. In one embodiment, the same carbohydrates used in the commercial vaccine could be linked to the L-PNA strands of the L-PNA:PNA(GAP). In practice, different carbohydrates could easily be attached to different L-PNA sequences. Using the instant L-PNAs, seven, or even more, different carbohydrates could be attached to the L-PNA:PNA(GAP) if desired to increase the number of strains represented in the vaccine.

Other vaccines could similarly be made by appending agents that resemble a disease-causing microorganism, such as a weakened or killed forms of the microbe or its toxins to a PNA. Common vaccines include, but are not limited to, various strains of influenza vaccines, various strains of hepatitis vaccines, cholera vaccine, bubonic plague vaccine, polio vaccine, yellow fever vaccine, measles vaccine, rubella vaccine, tetanus vaccube, diphtheria, vaccine, mumps vaccine, typhoid vaccine, tuberculosis vaccine and rabies vaccine. Such vaccines could be produced by appending the appropriate agent to a PNA of the instant invention that could then be used to form a L-PNA:PNA(GAP) macromolecule. Vaccines to other disease states can be produced by attaching the appropriate agent to a PNA. Additionally, vaccines to multiple conditions can be made by appending multiple agents to a PNA that could then be used to form a L-PNA:PNA(GAP) macromolecule.

Provided herein are methods of treating or inhibiting a disease state in a mammal by administering to the mammal a therapeutically effective amount of one or more described L-PNA:PNA(GAP) macromolecules having a substituent capable of binding a cell surface protein. In some embodiments the cell surface protein is a transmembrane protein, lipid-anchored protein, peripheral protein, a cellular receptor or an adhesion protein. In some aspects the mammal can be a rodent (mouse or rat), equine, feline, canine, or primate. In some embodiments, the described primate can be a human.

As used herein, the phrase “capable of binding a cell surface protein” refers to a substituent that can be a ligand for the cell surface protein. Any substituent that can be or is a ligand for a cell surface protein by itself (i.e., when the substituent is not bound to a L-PNA:PNA(GAP) macromolecules) is considered to be a suitable substituent in this context.

In some embodiments, the described methods are therapeutic methods directed to reducing metastasis in a mammal. In some embodiments these methods are carried out by administering to the mammal a therapeutically effective amount of one or more described L-PNA:PNA(GAP)s having a substituent capable of binding a cell surface protein. In particular embodiments the therapeutic agent administered to reduce metastasis is a L-PNA:PNA(GAP) having 15 cyclo-RGD gamma substituents. In some aspects the mammal can be a rodent (mouse or rat), equinee, feline, canine, or primate. In some embodiments, the described primate can be a mouse. In some embodiments, the described primate can be a human.

Also described are methods for detecting the presence of a cell surface protein using the described scaffolds. In some embodiments the described method can be carried out by administering to a subject a described L-PNA:PNA(GAP) capable of binding to the cellular protein target of interest and detecting the administered L-PNA:PNA(GAP). To facilitate detection, in some embodiments the L-PNA:PNA(GAP) may be labeled with a reporter molecule. For example, the L-PNA:PNA(GAP) may be radio labeled, conjugated to a fluorescent label, biotinylated, conjugated to DOTA, DTPA, or consist of a radionuclide. Other acceptable labels are widely known within the art. In some aspects the subject may be a rodent (mouse or rat), equine, feline, canine, or primate. In some embodiments, the described primate can be a human.

Also as used herein, the description of one or more method steps does not preclude the presence of additional method steps before or after the combined recited steps. Additional steps may also be intervening steps to those described. In addition, it is understood that the lettering or order of process steps or ingredients is a convenient means for identifying discrete activities or ingredients and the recited lettering can be arranged in any reasonable sequence.

Where a range of numbers is presented in the application, it is understood that the range includes all integers and fractions thereof between the stated range limits. A range of numbers expressly includes numbers less than the stated endpoints and those in-between the stated range. A range of from 1-3, for example, includes the integers one, two, and three as well as any fractions that reside between these integers.

The following examples further illustrate the invention but, of course, should not be construed as in any way limiting its scope.

### EXAMPLE 1

This example demonstrates the generation of an initial library of ligand-modified PNA conjugates and the multivalent landscape of the conjugates in accordance with an embodiment of the invention.

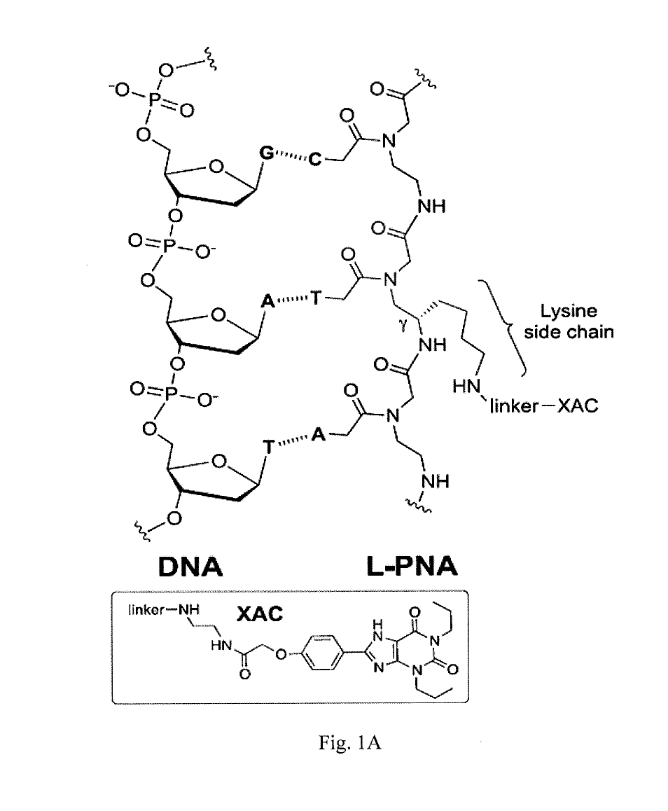

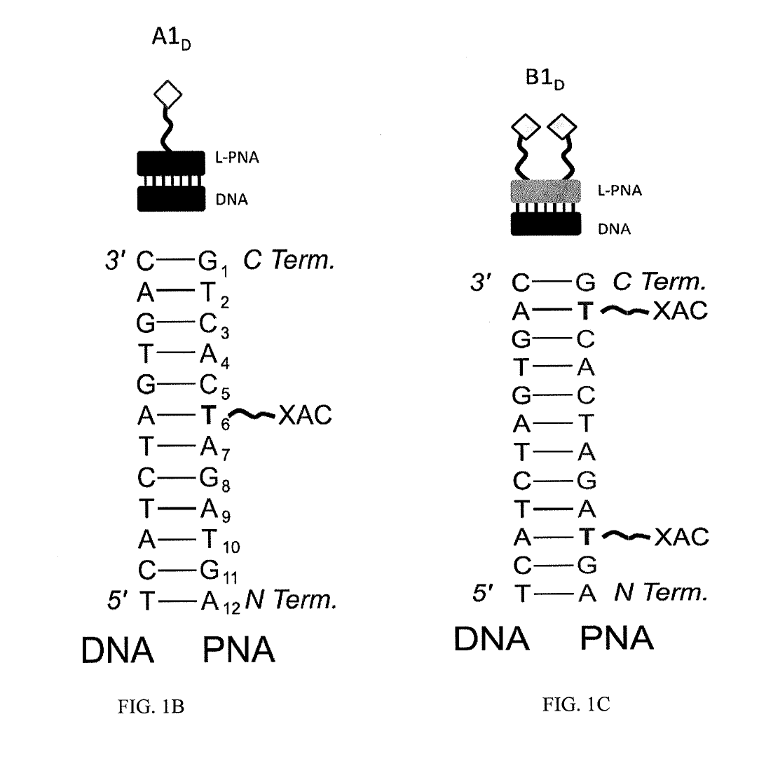

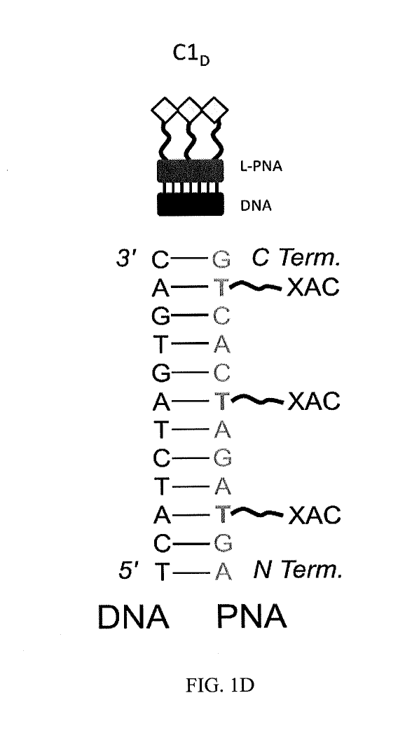

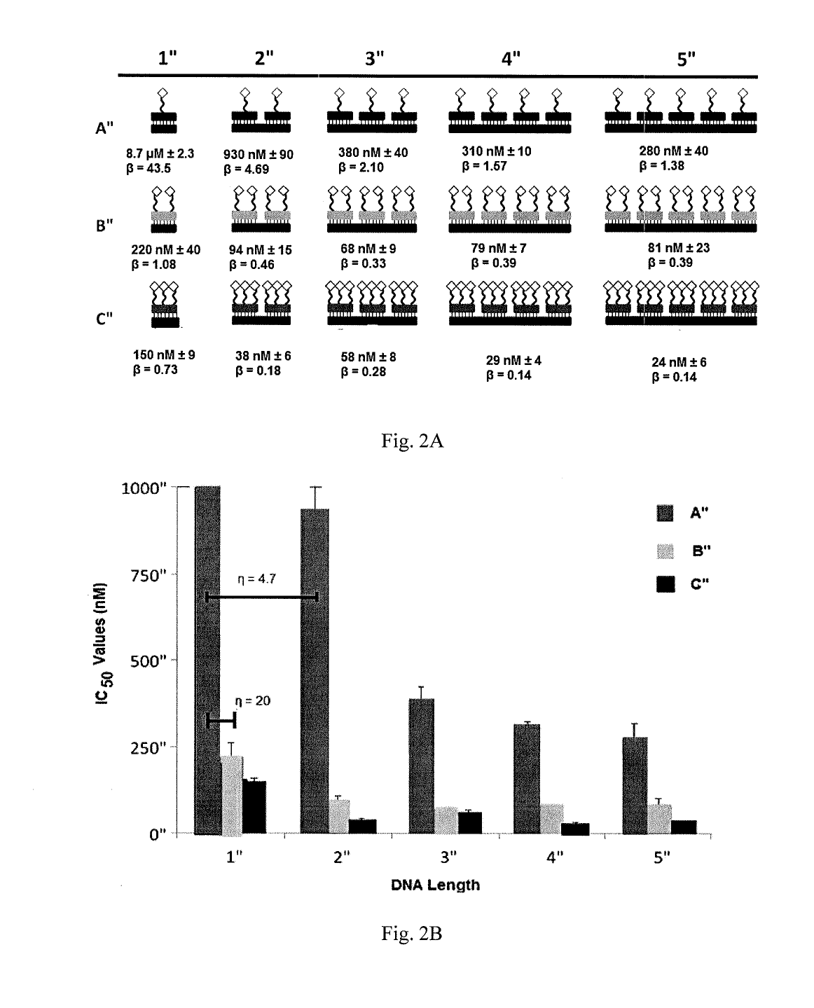

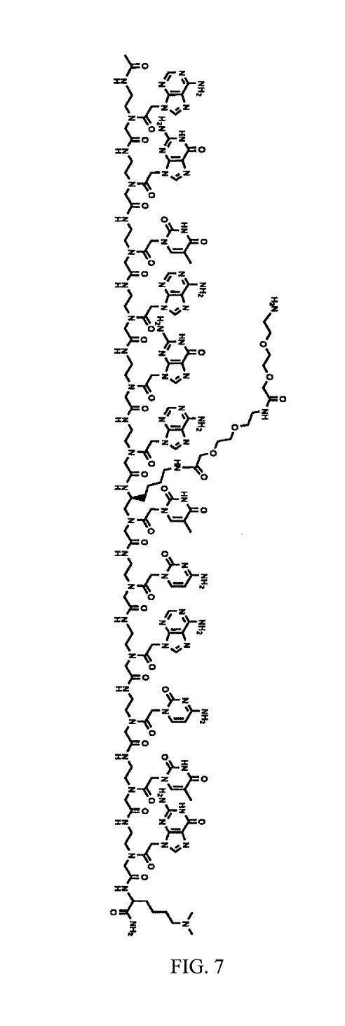

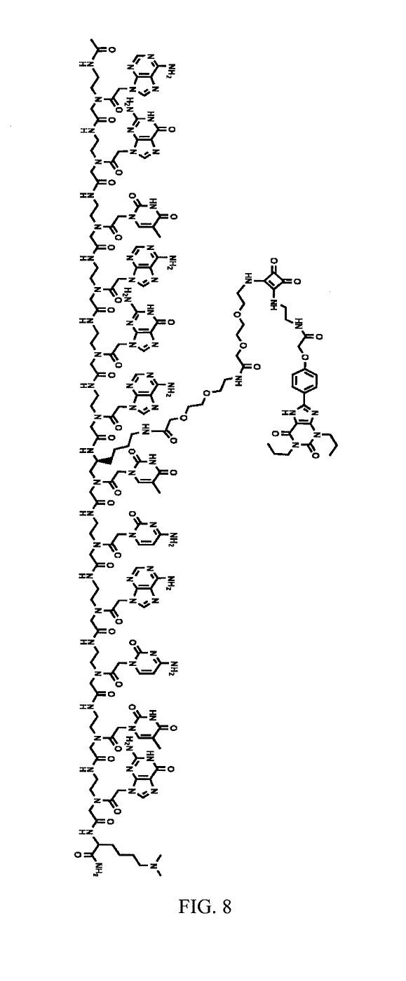

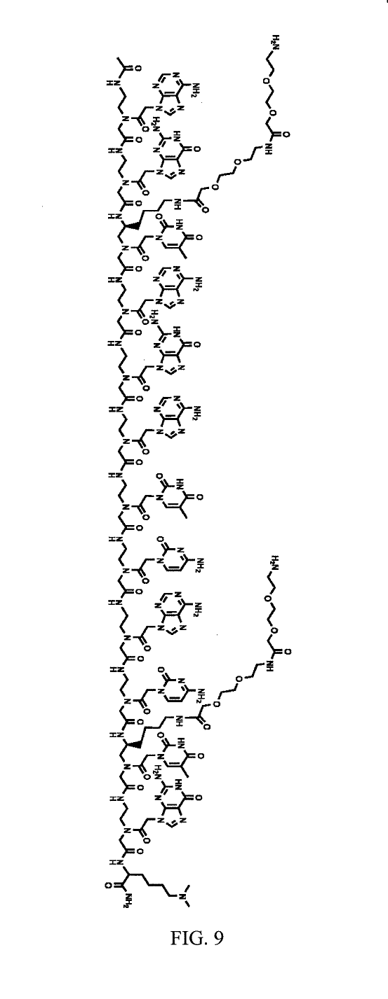

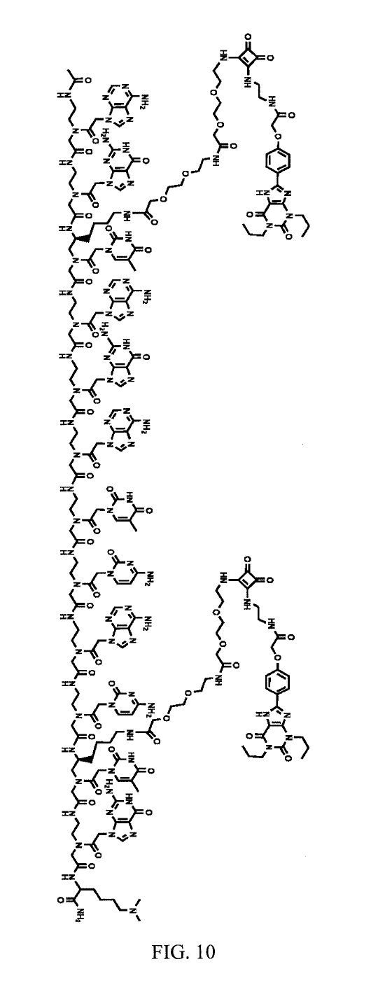

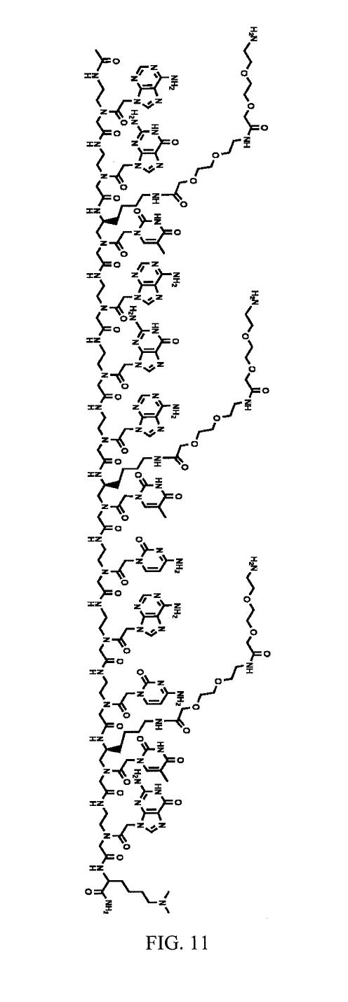

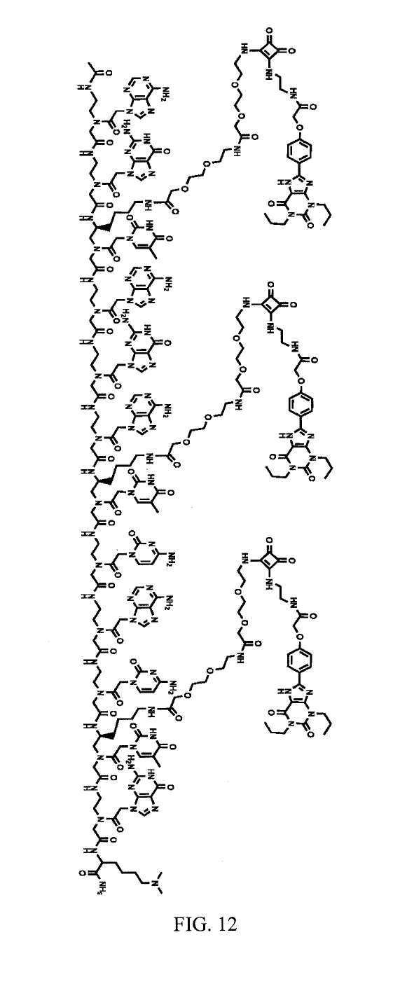

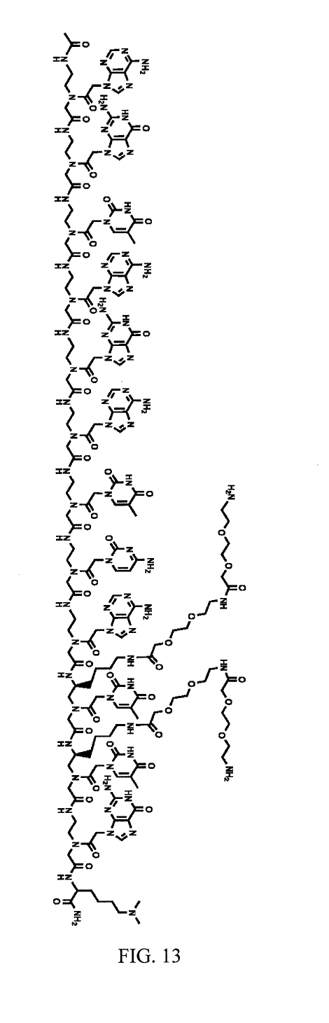

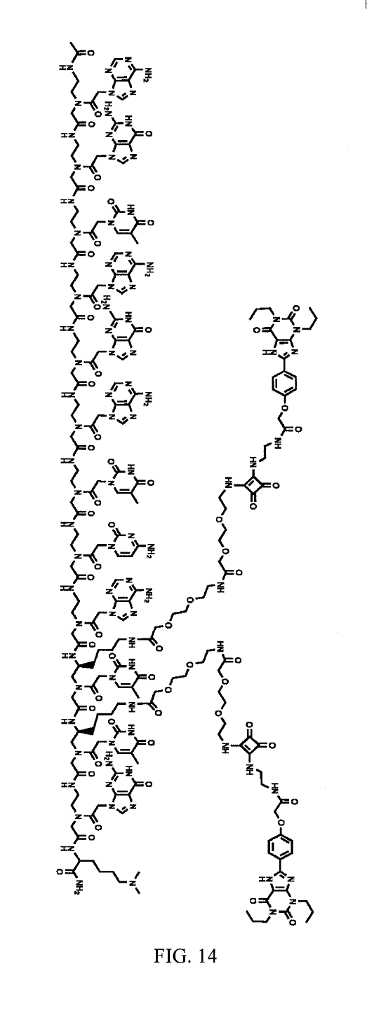

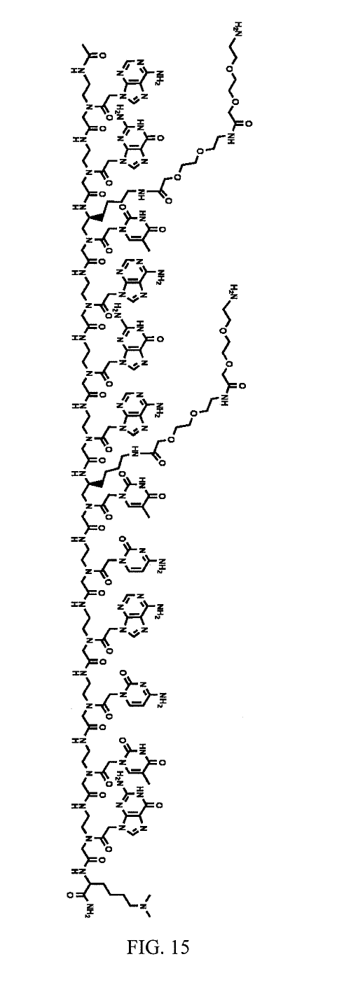

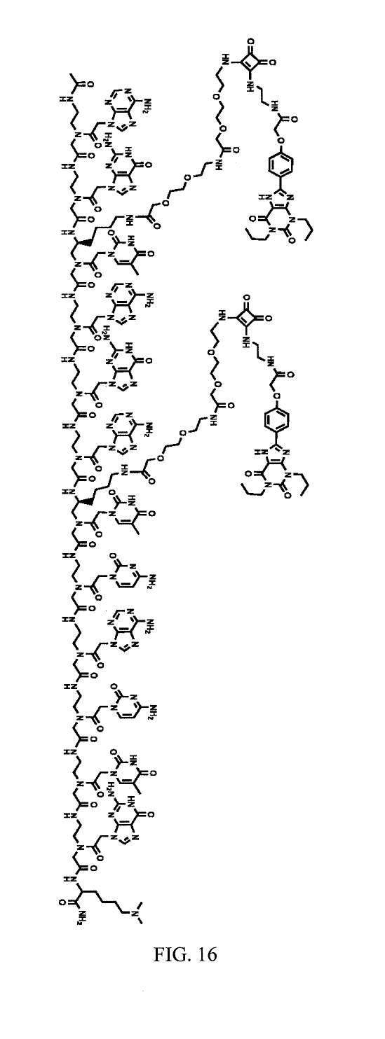

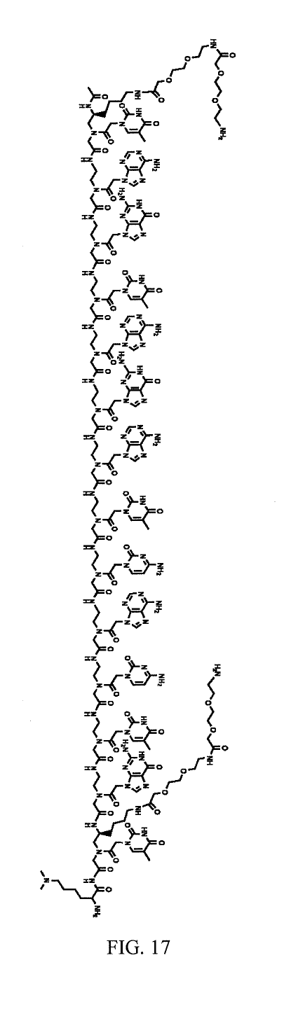

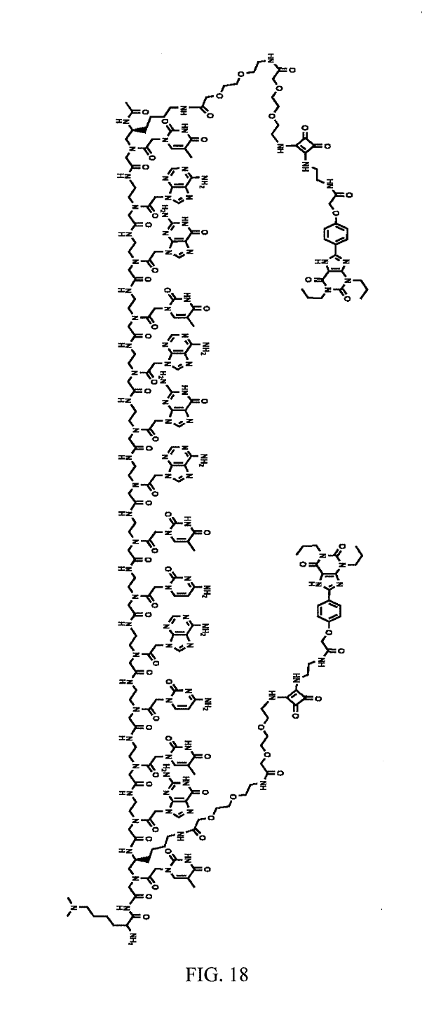

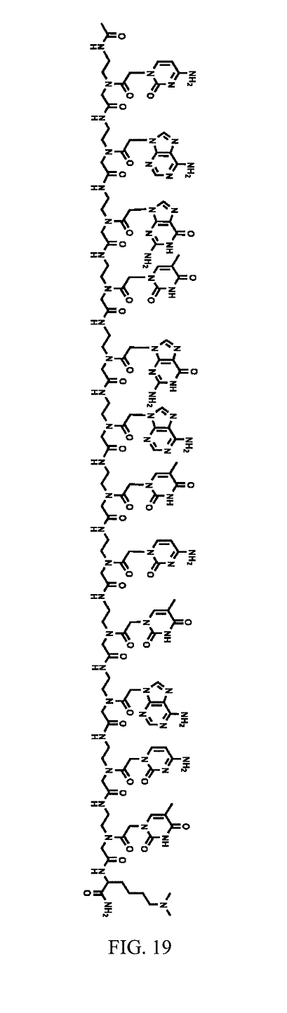

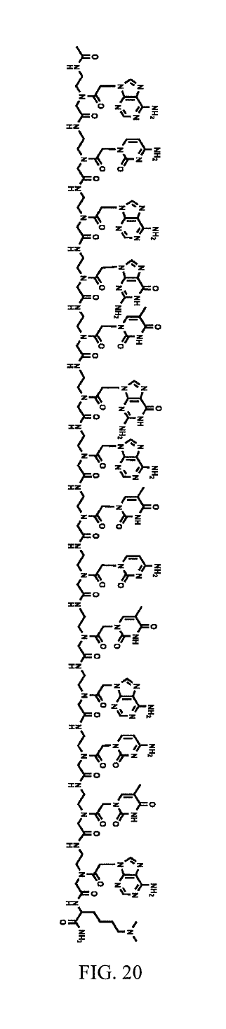

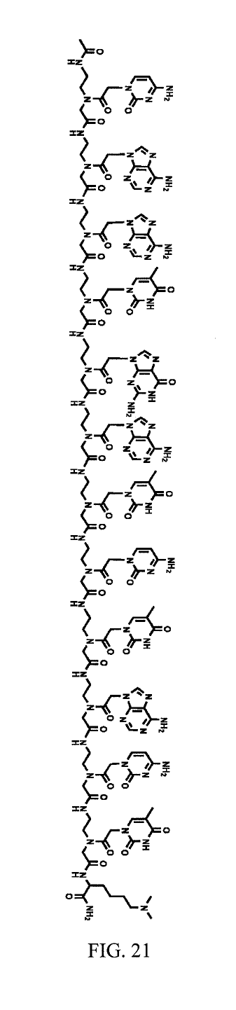

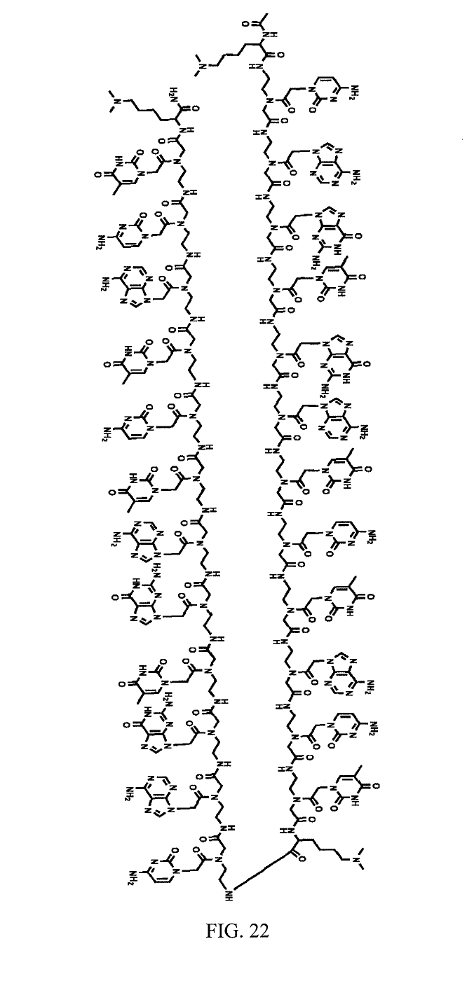

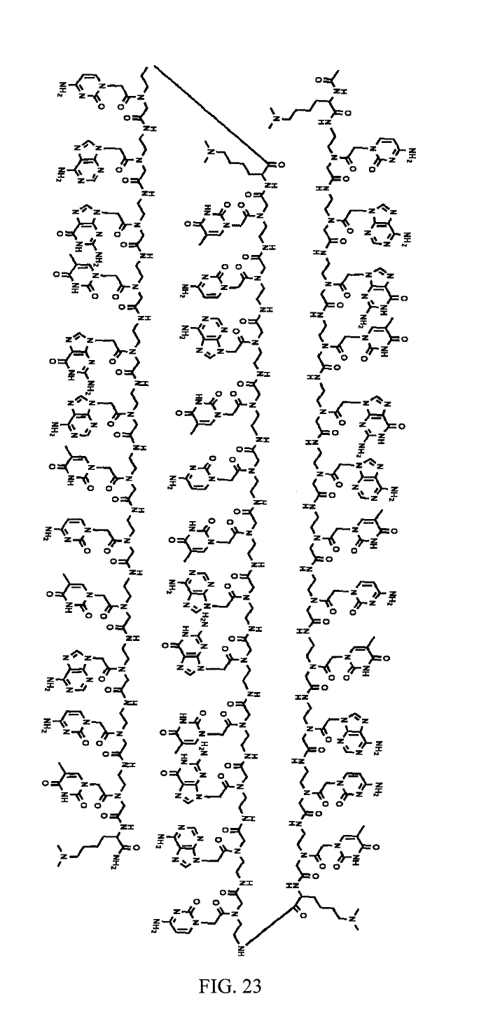

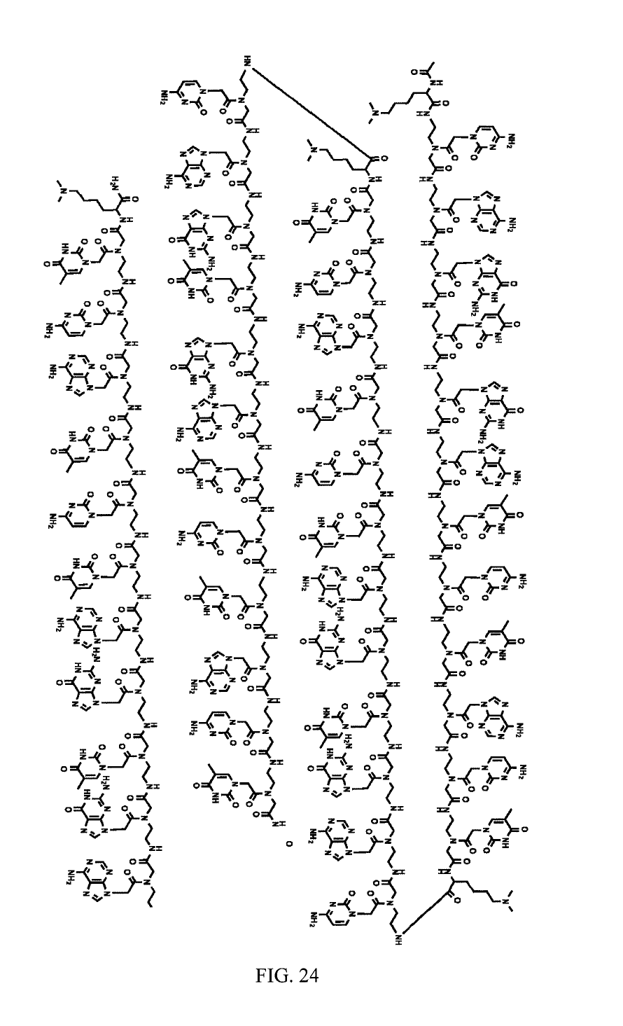

To generate a multivalent library of ligand-modified PNA conjugates (L-PNAs), a high affinity AR antagonist, xanthine amine congener (XAC), was conjugated to PNA oligomers via a γ-sidechain derived from lysine (γ-Lys) (FIG. 1A). Ligands attached to this sidechain within an L-PNA oligomer do not interfere with the ability of the L-PNA to bind to complementary DNA sequences by traditional Watson-Crick base pairing. A series of PNA oligomers, each consisting of 12 nucleobases, was synthesized in which one, two, or three γ-Lys sidechains were incorporated into the sequence (FIGS. 1B-1D). The primary amines at the ends of the γ-Lys sidechains serve as the attachment points for the XAC ligands. Two mini-PEG (8-amino-3,6-dioxaoctanoic acid) linkers inserted between the amine and the XAC minimize steric repulsion with the receptor protein (FIGS. 7-18). Three L-PNAs were generated in this manner, each containing 1, 2, or 3 XAC ligands, referred to as types A, B, and C respectively (FIGS. 1B-1D). Annealing each L-PNA to complementary DNA sequences designed to bind 1 to 5 L-PNAs generates a library of multivalent L-PNA:DNA duplexes (FIG. 2A). Overall, 15 complexes were generated (3 different PNAs complexed to 5 different DNAs) to systematically span a ligand valency between 1 and 15 XAC ligands. Each L-PNA:DNA complex is named according to its individual components. For example, a type A construct bearing 3 L-PNA units along the DNA backbone is referred to as A3_{D}, which contains 3 ligands (see FIG. 2A).

Each, member of the library was tested for binding affinity using an established A_{2A}AR membrane-based radioligand inhibition assay to explore the effects on protein binding of increasing the ligand valency and density. Although such protocols have become standard in the investigation of GPCR behavior, membrane binding assay data can be complicated by the presence of multiple protein binding states. These binding isotherms are a composite of these states, and special cases can highlight multiple binding thresholds (i.e. IC₅₀ or Kᵢ values). However, they are typically observed as a monophasic binding isotherm. Only monophasic isotherms were observed, which provide a single binding affinity for each compound. These affinities are presented in FIG. 2A and in the multivalent landscape plotted in FIG. 2B.

One way to analyze data from a multivalent screen is to calculate the (β-parameter for each member of the library (β=K_{d} (L-PNA:DNA)/K_{d} (monomeric ligand)), where β describes the benefit of the multivalent scaffold relative to the monovalent ligand, and lower values signal enhanced binding due to multivalent effects. Calculated β values for each member of the multivalent library, are shown below the IC₅₀ values in FIG. 2A. These values reveal some important features. While the attachment of one ligand to the L-PNA:DNA (A1_{D}) scaffold lowers the binding affinity compared to the ligand alone, the addition of more ligands to the scaffold quickly overcomes any loss in binding. This observation signals a multivalent effect. While the β values identify the most potent binders in the library, the patterns of improvements in affinity over the entire dataset indicated that different types of multivalent effects occur as the number of ligands increases.

Therefore, a new method was developed to analyze the results from the multivalent screen. The parameter η is defined as the change in binding affinity between any two L-PNA:DNA complexes when the change in ligand valency is normalized. When comparing two complexes, η values of approximately 1 indicate that individual ligand binding affinity is roughly the same and that any improvements in binding are due solely to the integral increase in the number of ligands. Values of η greater than 2 suggest a statistically significant increase in the individual ligand binding affinity that exceeds the expected improvement from simply having more ligands. When examined in this manner, the multivalent landscape in FIG. 2B indicates that most η values are near 1. However, η values greatly exceed 1 when comparing A1_{D} to either A2_{D} (η=4.7, p=0.016) or B1 _{D} (η=19.8, p=0.012). The main conclusion from this analysis of the multivalent landscape is that the most significant improvements in ligand-receptor binding occur when moving from a valency of 1 to 2 ligands.

### EXAMPLE 2

This example demonstrates ligand spacing on binding of a L-PNA:DNA complexes in accordance with an embodiment of the invention.

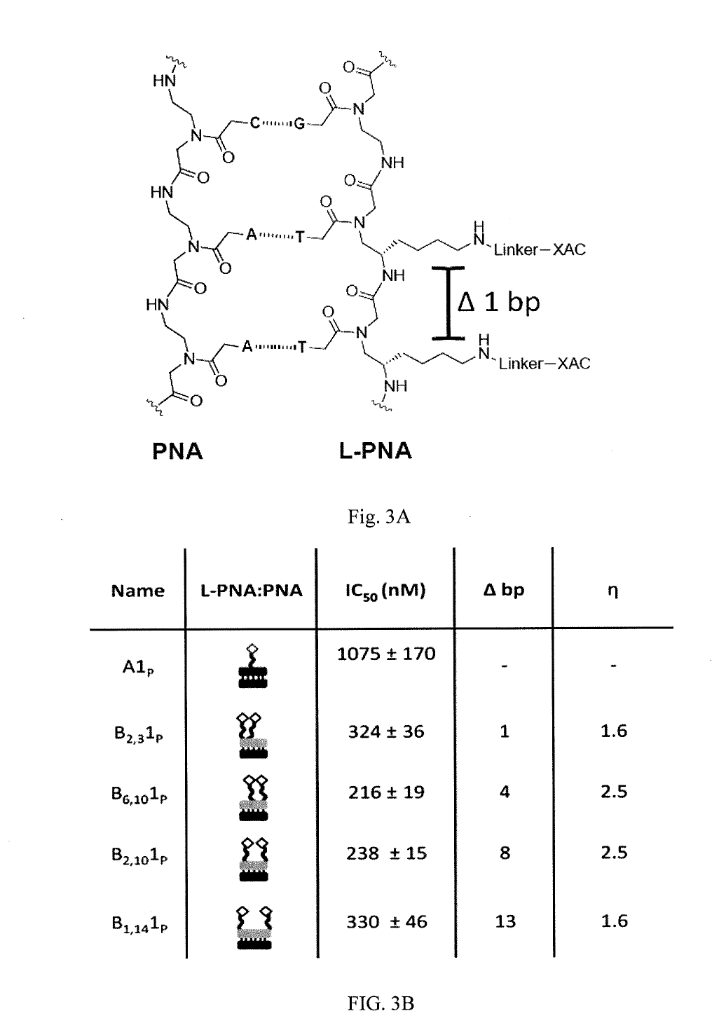

The initial results obtained indicated that a L-PNA:DNA complex bearing two XAC ligands binds significantly better than a corresponding monovalent complex. Next the effects of ligand spacing were assessed. A series of bivalent constructs were examined where the two γ-Lys sidechains bearing XAC ligands were systematically shifted along the PNA backbone (FIGS. 3A-3B). To minimize the electrostatic influence of the negative charges on the DNA phosphodiester backbone, the DNA was replaced with a PNA that was complementary in sequence. It is well-established that PNA:PNA duplexes maintain traditional nucleobase pairings in double-helical structures. Experimental results revealed that DNA can have a negative effect on binding because A1_{P} was 8-fold more potent that A1_{D} (p=0.0015). With the exception of lysine residues added at the termini to promote aqueous solubility, the resulting L-PNA:PNA duplex is charge neutral and should not experience charge-charge repulsion with phosphate groups on the membrane containing the receptor. In total, four B1_{P} complexes were generated (B_{2,3}1_{P}, B_{6,10}1_{P}, B_{2,10}1_{P}) and B₁,₁₄10 with various distances between the ligands, where the sidechains on the L-PNA backbone were separated by 1, 4, 8, or 13 nucleobases (FIG. 3A).

The bivalent L-PNA:PNA complexes all bound with higher affinity to A_{2A}AR (η=1.6 to 2.5, p≧0.007) compared to A1_{P} (FIG. 3b). Within the series of bivalent constructs, the narrowest (B_{2,3}1_{P}) and the widest (B_{1,14}1_{P} complexes were the weakest binders. The B_{6,10}1 ₚ and B_{2,10}1 ₚ complexes bound with higher affinities, yet were experimentally indistinguishable from each other at this level (p≧0.05). Although less dramatic compared to the previous multivalent screen, the binding data and η values indicate that the strength of ligand binding in this series of bivalent complexes depends on the distance and angle between the sidechains that display the ligands.

Experiments were also conducted to assess the thermal stability of PNA:PNA complexes with PNA:DNA complexes. These experiments were performed in 600 μL cuvettes on Agilent 8453 UV-Vis Spectrophotometers. A 5 μM solution (225 μL) of PNA and DNA were prepared in TRIS (100 mM) and added to the cuvette. After warming at 90° C. for 5 min, the cuvette was cooled at a rate of 1° per min. The absorbance at 260 nm was plotted versus temperature and curve fit to obtain melting temperatures. The results in Table 1 demonstrate that PNA:PNA complexes have greater thermostability than PNA:DNA complexes.

TABLE 1
Thermostability of nucleic acid complexes

| Duplex | Tm ° C. | Error |
|---|---|---|
| PNA:DNA | 57.20 | 0.06 |
| A1_{D} | 55.53 | 0.48 |
| B1_{D} | 54.54 | 0.45 |
| C1_{D} | 64.60 | 0.22 |
| A1_{P} | 72.07 | 0.13 |

### EXAMPLE 3

This example demonstrates a theoretical model and docking for a L-PNA:PNA in accordance with an embodiment of the invention.

Based on the data from the multivalent screens, it seemed likely that bivalent complexes bind to homodimeric pairs of A_{2A} receptors. To investigate this possibility in more detail, a coarse-grained statistical mechanics model was developed to interpret the experimental binding data in FIG. 3B and suggest the relative abundance of dimeric versus monomeric receptors. The model examines the relative ability of all 78 possible configurations of monovalent and bivalent sidechain combinations along the L-PNA:PNA backbone to bind to a theoretical receptor. The linker groups attached to the sidechains of the ligands are flexible thus the conformational states accessible to each sidechain were modeled as a polymer with a self-avoiding walk. The receptors were modeled as two concentric circles, an outer circle representing the excluded volume portion of the receptor and an inner circle representing its ligand-binding site. Ensembles of different receptor densities were placed in a two-dimensional plane representing the lipid bilayer of a membrane. Discrete ratios of receptor dimers and monomers were assigned, ranging from all monomers to all dimers. Each sidechain configuration of the L-PNA:PNA construct was examined for its binding potential to the receptor ensemble.

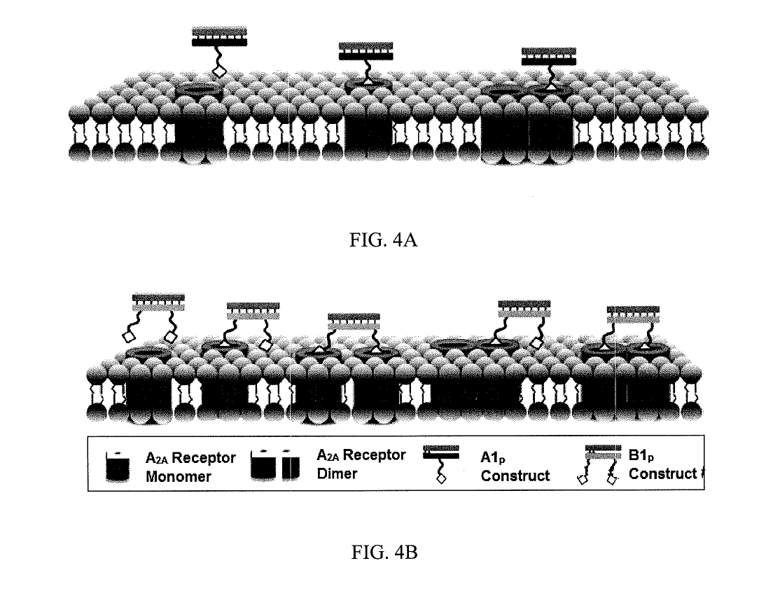

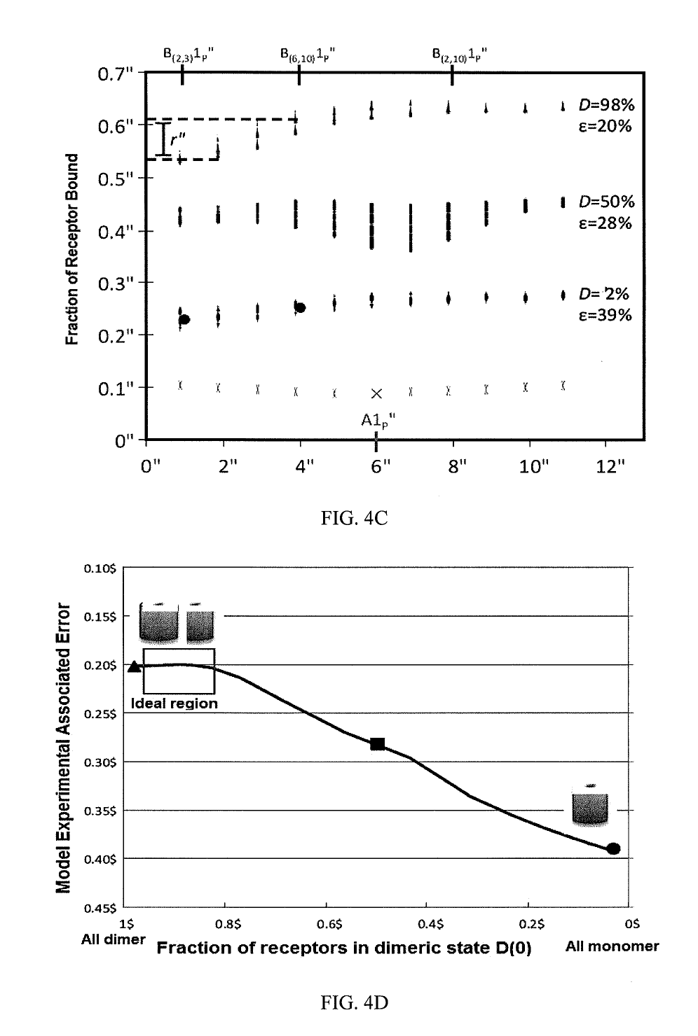

By assigning a fixed energy to each interaction, only a discrete number of different binding states exist between L-PNA:PNA and the receptor. Examples of these states for the monovalent (A_{1P}) and bivalent (B_{1P}) complexes are highlighted in FIGS. 4A and 4B. In this model, the enthalpy of ligand binding to the receptor was assumed to be the same for each state in which there is a binding event. Therefore, only the changes in entropy of receptor binding between the different L-PNA:PNAs were considered in the subsequent calculations. The model determines the probability of occurrence for each possible state, calculates the density of states for each protein ensemble, and subsequently provides a partition function with an energetic term (based on the entropy of binding) that represents the likelihood of receptor dimerization. Finally, the fraction of ligand-bound receptors in the ensembles is calculated for each L-PNA:PNA configuration. In FIG. 4C, some of these data are presented for four different data sets. Each dataset in the figure (▴, ▪, ) consists of the 66 different combinations of L-PNA:PNA bivalent complexes interacting with receptors at a discrete ratio of dimer to monomer (D). The x represents the 12 possible monovalent L-PNA:PNAs interacting with the receptors.

Depending on the percentage of receptor dimer (D) assigned in the model, there are clear differences in the predicted binding of bivalent L-PNA:PNAs. For instance, when only 2% of receptors exist as dimers (D=2%) there is a low fraction of bound receptors across the set of 66 possible bivalent L-PNA:PNAs (). If 98% of receptors are dimers (D=98%), then the predicted fraction of bound receptors is much higher (▴). These differences exist for bivalent L-PNA:PNA. For the 12 possible monovalent L-PNA:PNAs (x), there is no change in the fraction of bound receptors as the percentage of dimer increases because the single ligand binds equally to all states of the receptor regardless of whether it is a dimer or monomer.

The experimental data was compared with the theoretical model to estimate the percentage of receptor dimers. The red bars at the top of FIG. 4C show where the experimental bivalent L-PNA:PNAs from FIG. 3B align within the model's 66 possible L-PNA:PNA configurations. The next goal was to determine which dataset (▴, ▪, , or others) had the best fit with the experimental values. To make this evaluation, ratios of IC₅₀ values from FIG. 3B are directly compared to the ratios for the same L-PNA:PNA complexes in the model. An example of this ratio is represented by r in FIG. 4C, which is the ratio of IC₅₀ values for B_{2,3}1_{P} to B_{6,10}1_{P}. The r from experimental data is compared to the same ratio predicted by the model in each dataset. In total, there are six experimentally-determined r ratios derived from FIGS. 3A-3B that are compared to the analogous ratios in the different datasets of the model. Discrepancies between the experimental and theoretical values are assigned an error (ε). The magnitude of the error between experiment and theory was used as a guide to assign the most likely percentage of receptor dimer (FIG. 4D).

The analysis suggests that bivalent L-PNA:PNA binds to A_{2A} receptors that exist as dimers. A model where the receptors exist largely as monomers does not account for the observed experimental data (ε≧40%). The best overlap between experiment and theory lies in the realm of 80-95% of receptors existing as dimers (see “ideal region” in FIG. 4D) and the remaining portion as monomers (ε≦20%).

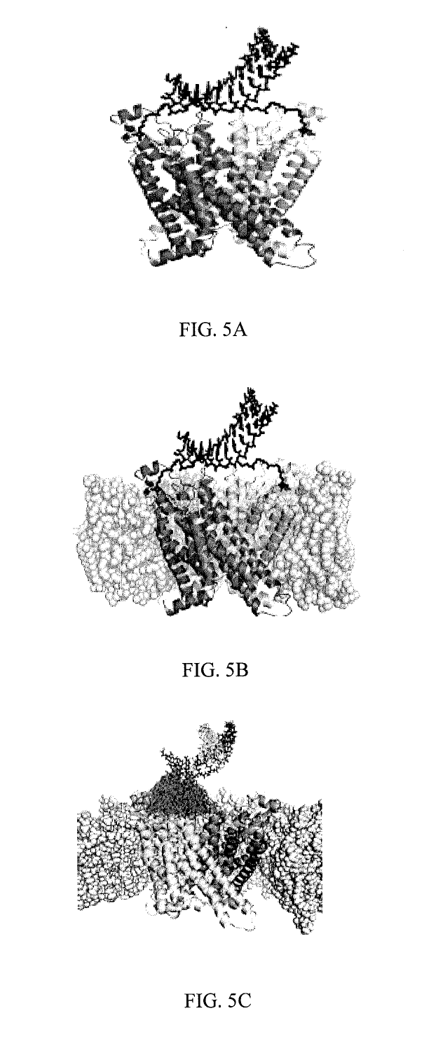

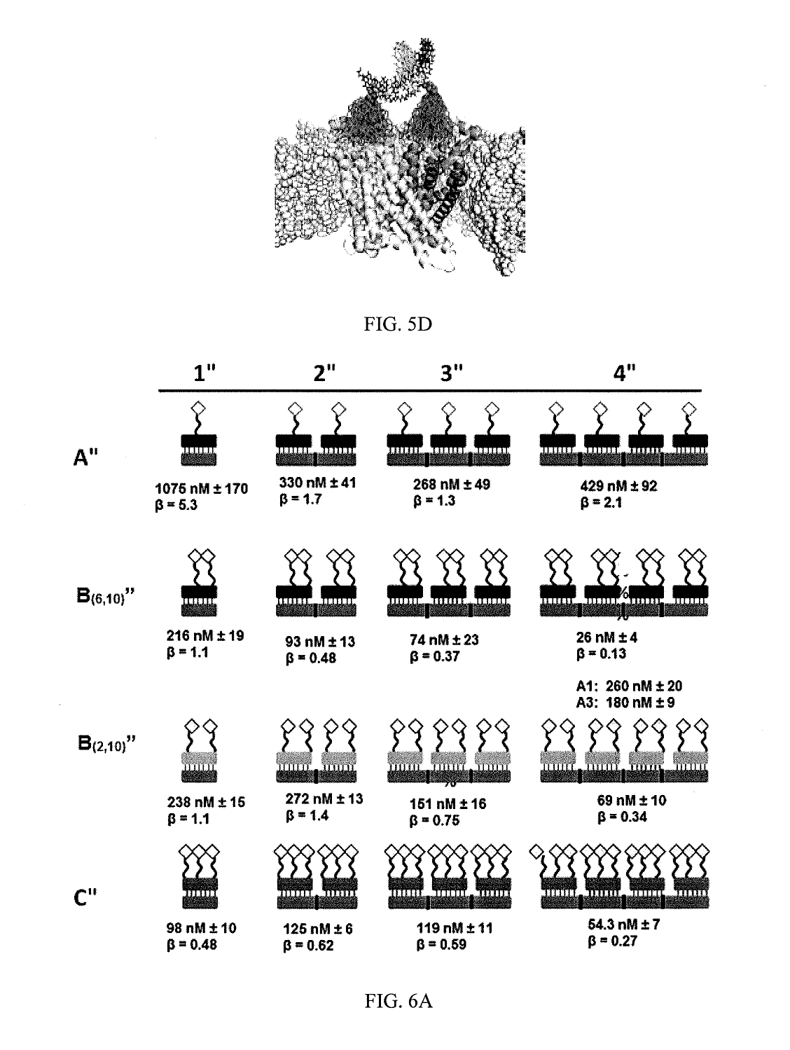

A molecular model further demonstrates that a bivalent L-PNA:PNA could bind to a dimer of A_{2A} proteins without excessive strain or clear steric clashing between the scaffold and the proteins. A dimeric A_{2A}AR protein was built and modeled to interact with B_{6,10}1 P (FIG. 5A). The structure of the A_{2A}AR monomers was based on a high resolution X-ray crystal structure (PDB 3REY) with XAC bound to the receptor (Dore, A. S. et al., Structure 19, 1283-1293 (2011)), and the likely contact regions between the protomers was determined through protein-protein docking. A PNA:PNA duplex model was created and connected to the bound XAC ligand through linkers that are identical to the ones used in the multivalent libraries. The construct was then optimized to an energy minimum and is displayed with (FIG. 5A) and without (FIG. 5B) the membrane. Both the molecular and statistical models suggest that the linkers are sufficient in length to allow access to both binding sites with an optimal sidechain placement. Additionally, the duplex backbone has ample space to hover over the protein surface without steric repulsion. These models represent a static snapshot of binding. A clearer representation of the flexibility associated with the sidechains is shown in FIGS. 5C and 5D. Models of the proposed A_{2A}AR dimer are overlaid with two bivalent L-PNA:PNA complexes. A subset of the sidechain conformations from the statistical model is displayed. As seen in FIG. 5C, the sidechains of B_{2,3}1_{P} do not overlap very well to simultaneously interact with both binding sites of the proposed A_{2A}AR dimer. In B_{6,10}1_{P} (FIG. 5D), the sidechains are more favorably arranged to simultaneously bind the dimer. This matches our experimental data; B_{6,10}1_{P} binds with higher affinity to A_{2A}AR than B_{2,3}1_{P} (IC₅₀ values of 216 nM versus 324 nM, FIG. 3B).

### EXAMPLE 4

This example demonstrates a L-PNA:PNA multivalent landscape, in accordance with an embodiment of the invention.

Comparing results from L-PNA:DNA and L-PNA:PNA demonstrated that the DNA can have a detrimental effect on binding to the receptor at low valencies. Bivalent L-PNA:PNA duplexes were used to examine the effects of intraligand distances on binding to A_{2A}AR. This approach was extended to higher valencies using longer PNAs as a replacement for DNA. Therefore, a modified PNA construct that can be made up to 48 bases in length was developed to support the binding of up to four complementary L-PNAs (with each L-PNA having between one and three sidechains bearing a XAC ligand). A second library containing 16 L-PNA:PNAs was constructed and used to generate a multivalent landscape by determining the binding affinity for each member of the library.

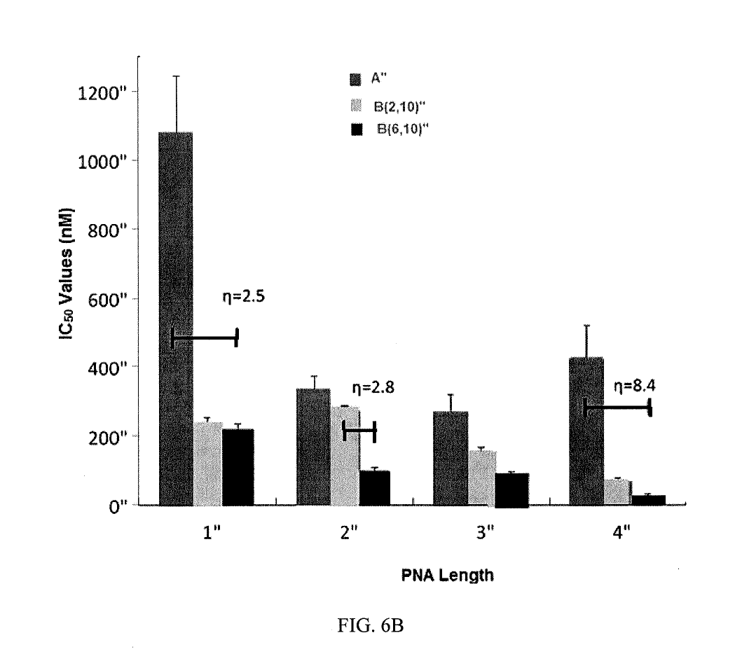

The multivalent library for L-PNA:PNA is shown in FIG. 6A, spanning valencies from 1 to 12 XAC ligands. The multivalent effects of two different bivalent type B PNAs were also explored in this library. Previously, B_{6,10}1 P and B_{2,10}1 P showed experimentally-indistinguishable binding affinities to A_{2A} when examined as a 1:1 L-PNA:PNA complex (FIG. 3B). With a better understanding of the likelihood for A_{2A} receptors to form dimers, it was particularly interesting to see if these constructs would show enhanced binding at higher valencies.

The results of screening this new L-PNA:PNA library are presented in FIG. 6B. Similar to the original multivalent screen, there is a significant enhancement of ligand binding efficiency when comparing valencies of one to two ligands (η=2.5) and, for the most part, all other improvements in binding affinity can be accounted for by the corresponding increase in ligand valency (η≅1). Remarkably, there is one data point in the multivalent landscape that is distinctly different: B_{6,10}4_{P} has a binding affinity that is markedly better than any of its surrounding neighbors (β=0.13). This specific L-PNA:PNA has a valency of eight XAC-bearing sidechains, arranged by pairs on 4 L-PNAs that are bound to a complementary PNA sequence containing 48 bases. The interligand spacing on the bivalent B_{6,10} PNA should be optimized for binding to an A_{2A} dimeric pair as shown previously (FIG. 3B). A highly-similar complex with identical size and valency (namely B_{2,10}4_{P}) binds significantly weaker (3 fold, (β=0.34), as do other L-PNA:PNAs with lower or higher valencies. A closer examination of the data series for B_{6,10} shows sequential improvement in binding affinity as successive additions of the complementary PNA are incorporated (with regard to IC₅₀ values, B_{6,10}1_{P}>B_{6,10}2_{P}>B_{6,10}3_{P}>B_{6,10}4_{P}). Interestingly, the same series with B_{2,10} does not show the same successive improvements in binding to A_{2A}. Further studies with B_{6,10}4_{P} show that it retains antagonist activity in a functional assay, and it has selectivity for A_{2A} receptors over Aₗ and A₃ that significantly exceeds that of the monovalent XAC ligand (FIG. 6A and Table 2). These results all suggest that B_{6,10}4_{P} has the proper dimensions and interligand spacing to bind simultaneously to multiple dimeric pairs of A_{2A} receptors.

TABLE 2

| | XAC Literature | B_{(6,10)}4_{P} | Increase |
|---|---|---|---|
| A₁:A_{2A} | 1.4 | 10.2 | 7.2 |
| A₃:A_{2A} | 2.4 | 6.9 | 2.9 |

A tenant of multivalency is the increase in selectivity of otherwise nonselective ligands. The binding affinity of B(_{6,10})4_{P} in A_{2A} overexpressed membranes was compared to AR homologues of Aₗ and A₃. The selectivity of the multivalent construct is compared to recently published literature values of XAC, which itself is nonselective for these receptors (Kecskes, et al., Bioconjug Chem 22, 1115-1127 (2011)).

### EXAMPLE 5

This example demonstrates the preparation and the dopamine D2 receptor activity of PNA-based multivalent nanoscaffolds, in accordance with an embodiment of the invention.

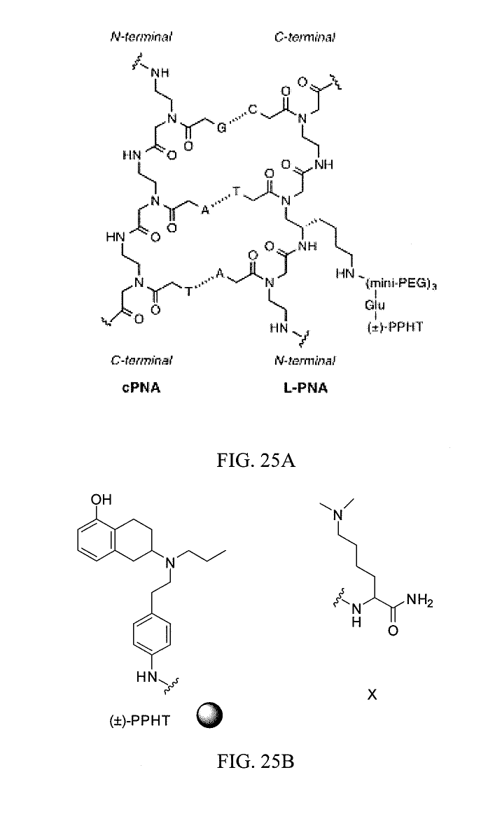

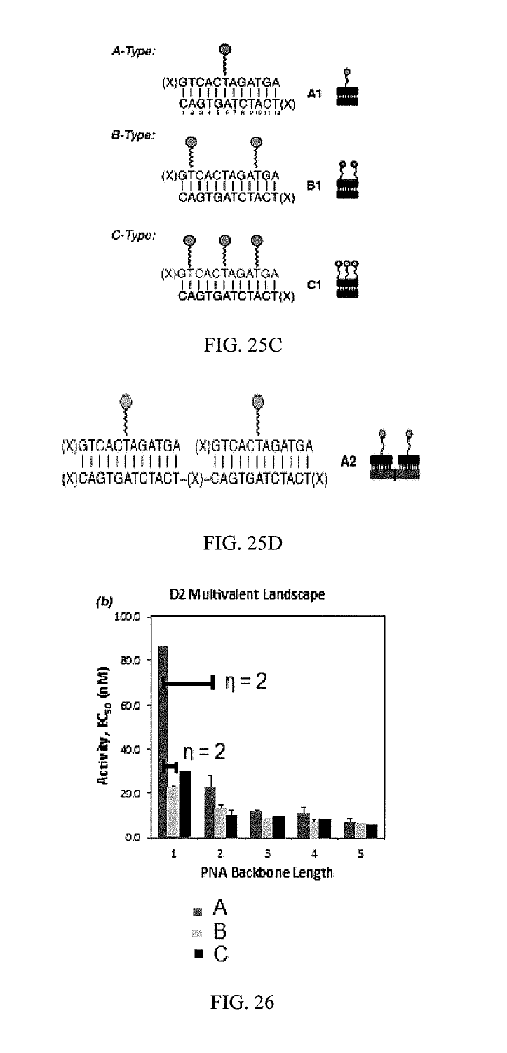

A library of ligand-modified peptide nucleic acids bearing a known D₂R agonist, (±)-PPHT (Soriano, A. et al., J. Med. Chem. 2009, 52, 5590-5602; Hacksell, U. et al., J. Med. Chem. 1979, 22, 1469-1475; Merali, Z. et al., Eur. J. Pharmacol. 1990, 191, 281-293; Bakthavachalam, V. et al., J. Med. Chem 1991, 34, 3235-3241) (FIG. 25B) was generated by systematic insertion of synthetic ^{L}Kγ monomers into a 12-residue PNA oligomer (FIG. 25A). To attach the ligand, the lysine moiety of the incorporated ^{L}Kγ monomer was extended from the main PNA backbone using three mini-PEG (8-amino-3,6-dioxaoctanoic acid) linkers. A glutamic acid modified (±)-PPHT was then conjugated to the mini-PEG N-terminus to generate the desired L-PNA. The ligand valency of L-PNAs was varied from 1 ligand per L-PNA (A-type), to 2 (B-type), and 3 (C-type) ligands per L-PNA by incorporating 1, 2, or 3 ^{L}Kγ-PNA monomers, respectively (FIG. 25C). In the A-type L-PNA constructs, the ligand was attached to the central residue, while in the B-type the ligands were attached at residues 2 and 6. The C-type constructs contained 3 ligands that were attached at residues 2, 6, and 10 (FIG. 25C). The L-PNAs were then annealed to complementary PNA oligomers (cPNA) in accordance with traditional Watson-Crick base pairing to provide a library of multivalent nanoscaffolds with defined valency, ligand spacing, and orientation (FIG. 25A). It has been demonstrated that L-PNA:PNA duplexes are preferred to L-PNA:DNA when targeting membrane proteins such as GPCRs. This preference is likely due to the minimization of the charge repulsion forces that exist between the anionic DNA backbone and the cell surface in the case of L-PNA:DNA. To identify the library constructs, each L-PNA sequence is referred to according to the constituent parts; for example, a single A-type L-PNA annealed to its 12-residue cPNA is referred to as A1 (FIG. 25C). Similarly, an A2 complex contains 2 A-type L-PNA units annealed along a 24-residue cPNA (FIG. 25D). In total, 15 unique L-PNA:PNA complexes were generated systematically and span a valency of 1-15 ligands (FIG. 26).

Each member of the L-PNA:PNA library was tested for D₂R activity using a whole cell β-arrestin recruitment assay (van Der Lee, M. M. et al., J. Biomol. Screen 2008, 13, 986-998;, R. B. et al., Mol. Pharmacol. 2014, 86, 96-105), and the data are summarized in Table 3 and FIG. 26. In general, the complexes were highly potent and demonstrated that an increase in ligand valency is associated with improved EC₅₀ values. Of particular interest was the dramatic change in the EC₅₀ values when the valency was increased from 1 to 2 ligands, specifically in going from A1 to A2, and A1 to B1. These data were further analyzed using η values, a term that was recently introduced (Dix, A. V. et al., J. Am. Chem. Soc. 2014, 12296-12303), to evaluate the change in D₂R activity between L-PNA:PNA complexes of the same type when the change in ligand valency is normalized (i.e. comparing sequential A-type L-PNA:PNA complexes). For the present purpose, an η value of approximately 1 indicates that improvement in D₂R activity are proportional to the increase in ligand valency. Alternatively, η values greater than 2 suggest that the incorporation of additional ligands results in an increase in D₂R activation that cannot be attributed solely to increased ligand content. Using the η parameter to analyze D₂R activity, an η value of 2 was obtained in the transition from 1 to 2 ligands for both the A1 to A2 and A1 to B1 transitions (FIG. 26). This indicates that increasing the valency from 1 to 2 ligands significantly enhances the D₂R activity. Interestingly, the ligand spacing in both the A2 and B1 constructs did not impact D₂R activity. In contrast, the addition of a third ligand to the 12-residue L-PNA C1 had a slightly detrimental effect on D₂R activation. This is likely due to steric crowding, which does not allow for favorable ligand-receptor interactions. The η values for the remaining constructs are close to 1, indicating that an increase in ligand valency beyond two ligands marginally improves D₂R activity. The nonspecific binding effects was also examined using an acetylated A type PNA that did not contain the (±)-PPHT ligand. Any nonspecific binding for this construct was not observed (data not shown). Taken together, these data demonstrate that the most significant activation of D₂R is observed when the ligand valency is increased from 1 to 2, suggesting that the formation of receptor dimers are important for D₂R activity. It is important to note that it is possible that the presence of the PNA construct drives dimer formation, and the receptor does not associate in the absence of ligand.

TABLE 3

| L-PNA | L-PNA Residues per cPNA | | | | |
|---|---|---|---|---|---|
| Type | 1 | 2 | 3 | 4 | 5 |
| A | 86.1 nM ± 0.1 | 22.3 nM ± 6 | 11.2 nM ± 2 | 10.2 nM ± 4 | 7.3 nM ± 2 |
| B | 22.3 nM ± 2 | 12.2 nM ± 1 | 8.0 nM ± 1 | 7.3 nM ± 0.8 | 5.8 nM ± 0.6 |
| C | 29.5 nM ± 0.9 | 10.2 nM ± 2 | 9.4 nM ± 0.5 | 7.8 nM ± 0.7 | 5.4 nM ± 0.7 |

The highly programmable and versatile nature of the PNA scaffold lends itself to the rapid assembly of multivalent tools in a predictable manner. The ability to rigorously and precisely control the ligand content, density, and spatial orientation of the PNA scaffold represents a clear advantage over traditional bi- and multi-valent approaches to investigate GPCRs. In this work, a multivalent nanoscaffold system based on L-PNA:PNA duplexes was used to explore the effects of multivalency on D₂R activity. A library of 15 unique L-PNA:PNA complexes bearing a known D₂R agonist, (±)-PPHT, was prepared, and the D₂R activity was evaluated. A significant increase in D₂R activity was observed when the valency was increased from 1 to 2 ligands in both the A1 to A2 and A1 to B1 constructs. Using values to further examine the A1 to A2 or B1 transitions, it was concluded that the substantial increase in D₂R activity is due to a multivalent effect that cannot be attributed solely to the change in ligand valency. The most likely explanation is that both ligands of A2 and B1 are bound to a dimer of D₂R. The incorporation of additional ligands in the remaining constructs improved activity proportionally to the increase in ligands. These data suggest that the formation of discrete receptor dimers are important for D₂R activity, but additional ligands do not significantly enhance signaling. With mounting evidence suggesting the importance of oligomeric GPCRs in disease pathophysiology, the L-PNA scaffold represents an important pharmacological tool to probe the effects of multivalent ligand displays on GPCR activity.

General Experimental Procedures and Materials

The following procedures and materials were used in the above experiments unless stated to the contrary.

Materials and Instrumentation. Commercial-grade reagents and solvents were used without further purification except as indicated. Boc-protected aegPNA monomers were purchased from PolyOrg, Inc. (Leominster, Mass., USA). HMBA Resin, 100-200 mesh, 1% DVB was obtained from Advanced Chemtech (Louisville, Ky., USA). Boc-mPEG was purchased from Peptides International (Louisville, Ky., USA). ^{L}Kγ-PNA Thymine Monomer was synthesized according to published procedures. The radioligands [³H]CGS21680 and [¹²⁵I]-AB-MECA were purchased from PerkinElmer (Waltham, Mass., USA), and [³H]R-PIA was purchased from Moravek Biochemicals (Brea, Calif., USA). All other reagents were obtained from Sigma-Aldrich (St. Louis, Mo., USA). PNA oligomer synthesis was performed on an Applied BioSystems 433A Automated Peptide Synthesizer. Purification of PNA oligomers was carried out using a X-Bridge Prep BEH 130 C18 5 μm (10×250 mm) column on an Agilent 1200s HPLC. The typical flow rate was 4 mL/min. HPLC solvents consisted of HPLC grade acetonitrile:MilliQ water (9:1) and 0.10% aqueous TFA. Wavelengths 220 nm, 260 nm, and 315 nm were monitored. High-resolution mass spectra (HRMS) were obtained on a LC/MSD TOF (Agilent Technologies, Santa Clara, Calif., USA). DNA oligomers were purchased from Integrated DNA Technologies, Inc. (Coralville, Iowa, USA) and used without further purification. UV quantification of PNA and DNA was performed using an Agilent 8453 UV-Vis Spectrophotometer.

Abbreviations. (ACN), acetonitrile; (Boc), tert-butoxycarbonyl-; (CGS21680), 2-[p-(2-carboxyethyl)phenyl-ethylamino]-5′-N-ethylcarboxamidoadenosine; (DMEM), Dulbecco's modified Eagle medium; (DCM), dichloromethane; (DMF), N,N-dimethylformamide; (DMSO), dimethylsulfoxide; (ESI-MS), electrospray ionization mass spectrometry; (HPLC), High Performance Liquid Chromatography; (I-AB-MECA), 4-amino-3-iodobenzyl)adenosine-5′-N-methyl-uronamide; (MBHA resin), 4-methylbenzhydrylamine resin; (NMP), N-methyl-2-pyrrolidinone; (mPEG), 8-amino-3,6-dioxaoctanoic acid; (PBS), phosphate buffered saline; (R-PIA), N⁶-[(R)-phenylisopropyl]adenosine; (PNA), peptide nucleic acid; (TEA), triethylamine; (TFA), trifluoroactic acid; (TfOH), trifluorosulfonic acid; (TRIS), tris(hydroxymethyl)aminomethane-hydrochloric acid buffered saline; (XAC), Xanthine amine congener; (ZM241385), 4-[2-[7-amino-2-(2-furyl)-1,2,4-triazolo[1,5-a][1,3,5]triazin-5-yl-amino]ethylphenol.

Preparation of PNA Oligomers. MBHA Resin (0.3 mmol/g) was prepared by swelling in DCM and downloading the resin with Boc protected N,N-dimethyl-L-lysine to 0.1 mmol/g capacity. PNA oligomers were made via solid-phase peptide synthesis in accordance with well-known procedures on either 5 or 25 μmol scale.

Sequences. Sequence used for XAC-conjugated PNA: AGT-AGA-TCA-CTG. Complementary antiparallel sequence: CAG-TGA-TCT-ACT. Note, for L-PNAs B(_{2,3)} and B(_{1,14)} the conjugated PNA sequences were modified to AGT-AGA-TCA-TTG and T-AGT-AGA-TCA-CTG-T respectively. The complementary sequences were adjusted accordingly.

General Resin Cleavage. Upon completion of PNA synthesis or solid phase coupling, the PNA-bound resin was transferred to a glass reaction vessel and washed with DCM, then TFA. The resin was swelled in TFA. The solvent was removed and a solution of m-cresol (150 μL), thioanisole (150 μL), TfOH (300 μL), and TFA (900 μL) was added and allowed to sit on the resin for 60 min. The solution was drained into a scintillation vial. This was repeated for a total of 3 washes, each time collecting the eluent in the scintillation vial. The pooled solution was concentrated, transferred to microfuge tubes, and precipitated using diethyl ether at a ratio of 1:10. The resulting flaky off-white solid was washed 3 times with diethyl ether and dried under vacuum. The resulting residue was diluted with 2:1 water:ACN and further purified on reversed phase HPLC.

General Conjugation Procedures. The XAC ligand (N-(2-aminoethyl)-2-[4-(2,3,6,7-tetrahydro-2,6-dioxo-1,3-dipropyl-1H-purin-8-yl)phenoxy]-acetamide, Sigma-Aldrich, St. Louis, Mo., USA) was conjugated to the free amino moiety on the PNA oligomer scaffold via squaric acid. This was performed by one of two methods:

1) Resin containing 5 μmol PNA with a free amine was swelled with NMP for 1 h in a glass reaction vessel. Following this, the solvent was drained and replaced with fresh NMP (500 μL). To this, triethylamine (300 μmol, 60 equiv) and 3,4-diethoxy-3-cyclobutene-1,2-dione (150 μmol, 30 equiv) were added. The vessel was sealed and agitated for 3 h. The resin was then washed (2×NMP, 2×DCM, and 2×NMP). A pre-dissolved solution containing XAC (100 ×mol, 20 equiv), 1:9 DMSO:NMP and TEA (150 μmol, 30 equiv) was added to the resin. The vessel was sealed and agitated until completion. In general, A-type L-PNAs were reacted for 18 h, while B and C L-PNAs were allowed 36 h to couple. The resin was then washed as previous and then conjugated PNA was cleaved from the resin.

2) In a 2 mL Eppendorf tube, lyophilized cleaved PNA was dissolved in minimal 2:1 anhydrous DMSO:ethanol. To this, triethylamine (60 equiv) and 3,4-diethoxy-3-cyclobutene-1,2-dione (30 equiv) were added. The vessel was flushed with nitrogen, sealed, and agitated for 3 h. The solution was concentrated and the residue was washed with diethyl ether (3×2 mL), and dried under vacuum to obtain the squaric acid-conjugated PNA intermediate as an off-white solid. A solution of XAC (20 equiv), 2:1 anhydrous DMSO:ethanol, and triethylamine (30 equiv) was added to the squaric acid-conjugated PNA intermediate. The vessel was flushed with nitrogen, sealed, and agitated until completion. In general, A-type L-PNAs were reacted for 18 h, while B and C L-PNAs were allowed 36 h to react. The solution was purified directly by reversed phase HPLC.

General HPLC Purification. PNA and conjugated-PNA residues were purified by reversed-phase HPLC using a 10×250 mm Waters XBridge prep BEH130 C18 5μm reverse phase column on an Agilent 1200s HPLC. Wavelengths 220 nm, 260 nm, and 315 nm were monitored. The typical flow rate was 4 mL/min. HPLC solvents consisted of ACN:water (9:1) and 0.15% aqueous TFA.

PNA residues were purified using one of the following methods:

Thermostat at 35° C. Gradient hold at 0% ACN 0-2 min, 10% ACN at 5 min, 20% ACN at 20 min, then wash with 100% ACN for 5 min.

Thermostat at 35° C. Gradient hold 0% ACN 0-1.9 min, 10% ACN at 2 min, 35% ACN at 25 min, then wash with 100% ACN for 5 min.

Thermostat at 50° C. Gradient hold 0% ACN 0-1.9 mM, 10% ACN at 2 min, 40% ACN at 35 min, then wash with 100% ACN for 5 min.

Quantification of PNA Oligomer Conjugates. Lyophilized PNA oligomers were dissolved in water. The absorbance of an aliquot was determined by UV-VIS spectroscopy after heating the sample for 5 min at 90° C. This was performed in triplicate. Using the extinction coefficient of the analogous DNA oligomer obtained from Applied Biosystems (Life Technologies, Grand Island, N.Y.), the concentration was determined.

General Annealing Condition for Formation of PNA:DNA or PNA:PNA Duplexes._RNA/DNAase free microfuge tubes, PNA, DNA and TRIS buffer (pH 7.5) were combined at room temperature. The final TRIS buffer concentration was 100 mM. Equivalents of PNA were added based on the number or repeating 12-mer sequence in the DNA. For example, to generate PNA:DNA multi5, a 5:1 molar ratio of PNA:DNA was used. The solution was heated to 90° C., held for 5 min, then slowly allowed to cool down to 25° C. over a period of 3 h.

LCMS Analysis of L-PNA:PNA Duplex. To confirm that the L-PNA:PNA complex was one species, and not an aggregate, mass spectrometry was utilized. Two complexes were analyzed and confirmed by this method: B2_{P} and B(_{6,10}4_{P}).

The PNA-complexes were separated from the monomers by reversed phase HPLC using electrospray ionization mass spectrometry (ESI MS) as the detection method. The HPLC was a Waters 1525u operated at a flow rate of 200 μL per min. Solvent A was 1% acetonitrile in water with 0.2% formic acid and 0.1% TFA. Solvent B was methanol with 20% acetonitrile with 0.2% formic acid and 0.1% TFA. The elution program starts at 0% B and is increased to 100% B in 9 min and finally held for 3 min at 100% B. The HPLC column was a Bruker-Michrom PLRP-S column with internal diameter of 2.1 mm and a length of 150 cm.

The ESI/MS was a Waters LCT Premiere operated in the positive ion V-mode. The ESI capillary voltage was 3.4KV. The multiple charged spectra were deconvoluted with MaxENT1.

B2_{P}. The components (B(_{2,10)} L-PNA and the complement PNA) were injected individually into the LC/MS system and their respective retention times and multiply charged ESI/MS spectra were recorded. The larger PNA (the complement) had a retention time of 8.99 min, the base peak was the 7+ion at 975.9 Da and a deconvoluted molecular weight of 6824. The smaller PNA (B(_{2,10)} L-PNA) eluted with a retention time of 8.49 min, the base peak was a 5⁺ ion at 699.1 Da and a deconvoluted molecular weight of 3490. The PNA-complex was observed with a retention time of 9.07 min and the individual components were simultaneously observed at that retention time. The ESI/MS of the lower mass component again showed a base peak at 1164.5 Da for the 3⁺ charged ion. The ESI/MS spectrum of the larger component yields the same molecular weight previously observed but the charge distribution is quite different with the base peak becoming the 6⁺ ion at 1138.4 Da. This change in charge state distribution is consistent with the larger PNA existing in a radically different state in the complex versus the monomeric form.

B(_{6,10)}4_{P}. The components (B(_{6,10)} L-PNA and the complement PNA) were injected individually into the LC/MS system and their respective retention times and multiply charged ESI/MS spectra were recorded. The larger PNA (the complement) had a retention time of 6.4 min, the base peak was the 8⁺ ion at 1719.3 Da and a deconvoluted molecular weight of 13764. The smaller monomer (B(_{6,10)} L-PNA) eluted with a retention time of 8.6 min, the base peak was a 4+ ion at 1307.6 Da and a deconvoluted molecular weight of 5226.4. The PNA-complex was observed with a retention time of 10.0 min and the individual components were simultaneously observed at that retention time. The ESI/MS of the lower mass component again showed a base peak at 1307.6 Da for the 4⁺ charged ion. The ESI/MS spectrum of the larger component yields the same molecular weight previously observed but the charge distribution is quite different with the base peak becoming the 10⁺ ion at 1375.8 Da. This change in charge state distribution is consistent with the larger PNA existing in a radically different state in the complex versus the monomeric form.

Cell cultures and membrane preparation. Chinese hamster ovary (CHO) cells stably expressing the recombinant hA₁ and hA₃ARs, and HEK293 cells stably expressing the hA_{2A}AR were cultured in Dulbecco's modified Eagle medium (DMEM) and F12 (1:1) supplemented with 10% fetal bovine serum, 100 units/mL penicillin, 100 μg/mL streptomycin, and 2 μmol/mL glutamine. In addition, 800μg/mL geneticin was added to the A_{2A} media, while 500 μg/mL hygromycin was added to the Aₗ and A₃ media. After harvesting, cells were homogenized and suspended in PBS. Cells were then centrifuged at 240 g for 5 min, and the pellet was resuspended in 50 mM Tris-HCl buffer (pH 7.5) containing 10 mM MgCl . The suspension was homogenized and was then ultra-centrifuged at 14,330 g for 30 min at 4° C. The resultant pellets were resuspended in Tris buffer, incubated with adenosine deaminase (3 units/mL) for 30 min at 37° C. The suspension was homogenized with an electric homogenizer for 10 sec, pipetted into 1 mL vials and then stored at −80° C. until the binding experiments. The protein concentration was measured using the BCA Protein Assay Kit from Pierce Biotechnology, Inc. (Rockford, Ill.).

Competitive Radioligand Binding to A_{2A} Receptors. Competition radioligand binding experiments were conducted to determine the binding affinities of PNA conjugates. A range of concentrations of PNA conjugates between 1 nM to 1000 nM was tested in competing for binding to A_{2A} receptors on cell membranes derived from A_{2A}-expressing HEK cells.⁴ Assay solutions (200 μL) in binding buffer (50 mM Tris-HCl, pH 7.5, 10 mM MgCl₂) comprised of cell membranes (100 μL), radioligand (50 μL), and PNA conjugates (50 μL) were prepared in test tubes, which were incubated at 25° C. for 1 h in a shaking water bath. Additionally, assay solutions containing either binding buffer instead of the PNA conjugates or 40 μM adenosine-5′-N-ethyluronamide were prepared for determining total and nonspecific radioligand binding to the membranes, respectively. The radioligand agonist [³H]CGS21680 was used for all A_{2A} experiments. [³H]R-PIA and [¹²⁵I]I-AB-MECA were used for Aₗ and A₃ binding experiments, respectively. After incubation, binding was terminated by rapid filtration through glass filter paper. The glass filter paper samples were then read by a scintillation counter (Tri-Carb 2810TR) to determine radioligand binding. The counts per ligand concentration were plotted and curve fit using Prism (GraphPad, San Diego, Calif., USA) to obtain IC₅₀ values. Each experiment provided a redundant data set and used 7 different concentrations of duplexed L-PNA. This was repeated in triplicate.

Fluorescent ligand binding experiments with flow cytometry (FCM). The HEK 293 cells expressing the hA_{2A}AR were grown in 12-well plates (approximately 200,000 cells/well) and incubated at 37° C. 36 h in the presence of 5% CO₂. When the confluency of the cells reached 80% (approximately 4×10⁵ cells/well), medium was replaced with fresh medium and B5_{Df} was added in the presence or absence of 10 μM ZM241385, and cells were processed for FCM. Note that BS_{Df} was generated using Alexa Fluor™ 488 labeled DNA purchased from IDT.

HEK 293 cells expressing A_{2A}ARs were incubated with different concentrations of B5_{Df} ranging from 1 nM to 50 nM for 30 min for a saturation binding experiments. To study binding kinetics, we incubated HEK293 cells expressing A_{2A}ARs with 30 nM B5_{Df} for different time intervals from 5 mM to 3 h. Nonspecific binding was measured in the presence of 10 μM ZM241385.

At the end of each time interval, the medium was removed and cells were washed two times with DPBS. After washing, 0.5 ml 0.2% EDTA solution was added to each well, and cells were incubated at 37° C. for 1 min. Following cell detachment, 0.5 ml medium was added to each well to neutralize the EDTA. The cell suspensions were transferred to polystyrene round-bottom BD Falcon tubes (BD, Franklin Lakes, N.J.) and centrifuged for 5 min at 23° C. and 400× g. After centrifugation, the supernatant was discarded, and cells were washed with 2 ml PBS and centrifuged again at 23° C. and 400 × g for 5 min. After discarding the supernatant, cells were suspended in 0.3 ml PBS and analyzed by FCM.

The intensity of fluorescence emission of each sample was measured by using FCM. Cell suspensions were vortexed briefly before analysis on a Becton and Dickinson FACSCalibur flow cytometer (BD, Franklin Lakes, N.J.) with excitation at 488 nm. Samples were maintained in the dark during the analysis to avoid photobleaching. MFIs were obtained in the FL-1 channel in log mode. Ten thousand events were analyzed per sample. Data were collected using Cell Quest Pro software (BD, Franklin Lakes, N.J.).

Association binding results were analyzed by fitting the binding data to a One-phase association equation y=y₀+(Plateau-y₀)(1-e^{kx}), where y₀ is the MESF (γvalue) when time (x value) is zero, Plateau is the MESF at infinite times, and K is the rate constant, expressed in inverse mM.

Saturation binding results were analyzed by fitting a One-site total and nonspecific binding equation to the binding data. Total and nonspecific binding was globally analyzed by fitting the total binding data to the equation y=BmaxX/(X+Kd)+(NS×X) and y=NS×X for the nonspecific binding data, where Bmax means the maximum specific binding in MESF units, Kd is the equilibrium binding constant in nM, and NS is the slope of nonspecific binding.

The measured fluorescence intensities were corrected with the subtraction of autofluorescence values of HEK 293 cells in the absence of any AR ligand.

Cyclic AMP Accumulation Assay. CHO cells expressing A_{2A}AR were seeded in 24-well plates and incubated at 37° C. overnight. The following day, the medium was removed and replaced with DMEM containing 50 mM HEPES, 10 μM rolipram, 3 U/mL adenosine deaminase, and increasing concentrations of a known agonist (CGS21680). The suspected antagonist (B_{(6,10)}4_{P})was added 20 min before the addition of agonist. The medium was removed, and the cells were lysed with 200 μL of 0.1 M HCl. One hundred microliters of the HCl solution was used in the Sigma Direct cAMP Enzyme Immunoassay following the instructions provided with the kit. The results were interpreted using a BioTek ELx808 Ultra Microplate reader (BioTek, Winooski, Vt.) at 405 nm.

Molecular Modeling of a PNA duplex bound to an A_{2A}AR homodimer

PNA:PNA Duplex:

Atomic-scale, computer models of select PNA-duplexes were developed with the QUANTA (Accelrys) and CHARMM software programs. The helical conformations were derived from the NMR solution structure of a gamma-methylated PNA-duplex 8-mer (PDB accession code: 2KVJ). Except for the nitrogenous bases, topologies needed to be developed for all the other molecular components. These were derived from the “all 27” set of topologies and parameters provided with the CHARMM program. Finally, all models were energy minimized with CHARMM to eliminate atomic overlap and optimize the bond lengths and angles.

hA_{2A}AR model:

To have a more complete 3D structure of the hA_{2A}AR, a model was built using the Homology Modeling tool implemented in the Molecular Operating Environment (MOE) suite and the available crystallographic data for this receptor subtype. The model was based on the highest-resolution hA_{2A}AR crystal structure (PDB ID: 4EIY) (Liu, W. et al., Science 337, 232-236 (2012)) where the template for the missing IL3 (from Lys209 to Gly218) was another inactive-state hA_{2A}AR crystal structure (PDB ID: 3REY) (Dore, A. S. et al., Structure 19, 1283-1293 (2011)). The intracellular C-terminal tail of the receptor (from Leu308 to Ser412) was not modeled, due to the absence of an useful template. Previously published FRET studies showed that the C-term does not participate in the A_{2A}AR homodimerization, and therefore seemed reasonable to exclude it from the modeling studies. The AMBER99 forcefield was used for protein modeling and the Protonate 3D methodology was used for protonation state assignment. The final model was refined through energy minimization until a RMS gradient of 0.1 kcal/mol Å. Model's stereochemical quality was checked using several tools (Ramachandran plot; backbone bond lengths, angles and dihedral plots; clash contacts report; rotamers strain energy report) implemented in the MOE suite.

Molecular docking of XAC-linker at the hA_{2A}AR model:

The XAC-linker structure was built using the builder tool implemented in the MOE suite and subjected to energy minimization using the MMFF94×force field, until a RMS gradient of 0.05 kcal/mol Å. Molecular docking of the ligand at the hA_{2A}AR model was performed by means of the Glide package part of the Schrodinger suite. The docking site was defined using key residues in the binding pocket of the hA_{2A}AR model, namely Phe (EL2), Asn (6.55), Trp (6.48) and His (7.43), and a 30Å×30Å×30Å box was centered on those residues. Docking of ligand was performed in the rigid binding site using the SP (standard precision) protocol. The top scoring docking conformations were comparable with the crystal pose of XAC at the A_{2A}AR (3REY) (Dore et al., supra). In particular, the main interactions observed in the crystal between the xanthine scaffold and the receptor were conserved, while the linker was pointing outside of the cavity making contacts with residues in EL2, such as Lys150, Lys153 and Gln157.

hA_{2A}AR homodimer model:

Homodimers were built starting from our hA_{2A}AR model and using the protein-protein docking tool of the ZDOCK server (ZDOCK 3.0.2). From the resulting poses, antiparallel dimers or poses not compatible with the nature of transmembrane proteins (i.e. excessive inclination or shift along the main axis between the two monomers) were discarded. For selected dimer poses, contact areas between two monomers were refined through energy minimization until a RMS gradient of 0.1 kcal/mol Å, using the AMBER99 forcefield implemented in the MOE suite.

Based on the monomers relative orientation we could identify, among the several reasonable poses returned by the software, two most populated clusters of dimers. The first cluster collected dimers with interface between TM5, TM6 and TM7, the second cluster dimers with interface between TM1, TM2 and helix 8. Both interfaces are comparable with some proposed through computational studies in a previous paper on A_{2A}AR homodimerization (Fanelli, F. & Felline, A. Biochim Biophys Acta 1808, 1256-1266 (2011)). For the majority of the possible dimers, the distance between the binding sites of the two monomers was 30-40Å. Considering that the distance between monomers was quite comparable among different dimers and that an unambiguous identification of the functionally relevant interface is very difficult, we selected one representative dimer belonging to the first cluster as starting point for the following modeling of the A_{2A}AR homodimer-PNA duplex construct.

A_{2A}AR homodimer-PNA duplex construct:

To combine the models of the hA_{2A}AR homodimer and of the PNA duplex the following procedure was performed. A XAC-linker structure was placed in its docked conformation inside each hA_{2A}AR monomer forming the dimer model. Then, the PNA duplex model was manually placed in proximity of the extracellular side of the dimer and the terminal groups of each XAC-linker structure were connected to the PNA chain at positions X and Y. Finally, the construct geometry was refined by energy minimization using the software MOE and the Amber12:EHT force field, until a RMS gradient of 0.1 kcal/mol Å. During the minimization the hA_{2A}AR dimer and the XAC scaffolds were kept fixed, the linker chains were free to move and the PNA duplex was considered as a rigid body.

PNA oligomer synthesis.

Commercial-grade reagents and solvents were used without further purification unless indicated. The resin (MBHA, 100-200 mesh, 1% divinylbenzene, 0.3 mmol g⁻¹, Advanced Chemtech) was prepared by swelling in CH₂Cl₂ and downloading the resin with N,N-dimethyl lysine to 0.1 mmoleg⁻¹ capacity. Boc-protected aegPNA monomers were purchased from PolyOrg. PNA oligomer synthesis was carried out on a 5 μmol scale on an Applied BioSystems 433A Automated Peptide Synthesizer. The resin was swelled with CH₂Cl₂ for 104 min before synthesis. The ^{L}Kγ-PNA monomer was synthesized according to published procedures.^{32,35} Activated ^{L}Kγ-PNA monomer was allowed 90 min to couple. A further treatment of trifluoroacetic acid deprotection solution was also used to remove the N-Boc protecting group from ^{L}Kγ-PNA residues. The lysine sidechains of ^{L}Kγ-PNA monomers (Fmoc) were orthogonally deprotected with 20% piperidine in DMF. When multiple ^{L}Kγ-PNA residues were present in the PNA oligomer (PNA-B and PNA-C; FIG. 3a), the primary amines on the sidechains were deprotected and coupled to mini-PEG residues in tandem, followed by coupling to (±)-PPHT. Purification of PNA oligomers was carried out using an XBridge Prep BEH 130 C18 5 μm (10 mm×250 mm) column on an Agilent 1100 HPLC. In all cases, 0.05% aqueous trifluoroacetic acid and acetonitrile were used as solvents.

General Annealing Conditions for Formation of L-PNA:PNA Duplexes.

In RNA/DNAase free microfuge tubes, L-PNA, cPNA, and PBS buffer were combined at room temperature. Equivalents of PNA were calculated based on the number or repeating 12-residue sequences in the PNA. For example, to generate L-PNA:PNA multi5, a 5:1 molar ratio of L-PNA:cPNA was used. The solution was heated to 90 ° C., held for 5 min, then slowly allowed to cool down to 25 ° C. over a period of 3 h.

β-arrestin Recruitment Assay.

Agonist-mediated recruitment of β-arrestin-2 was determined using the DiscoveRx PathHunter complementation assay (DiscoveRx Inc, Fremont, Calif.), as previously described (Free, R. B. et al., Mol. Pharmacol. 2014, 86, 96-105; Bergman, J. et al., Int. J. Neuropsychopharmacol. 2013, 16, 445-458). Briefly, CHO-K1 cells stably expressing the D₂R were seeded in cell plating (CP) media (DiscoveRx) at a density of 2625 cells/well in 384-well black, clear-bottom plates. Following 24 h of incubation, the cells were treated with multiple concentrations of compound in PBS buffer containing 0.2 mM sodium metabisulfite, and incubated at 37° C. for 90 min. DiscoveRx reagent was then added to cells according to the manufacturer's protocol followed by a 60 min incubation in the dark at room temperature. Luminescence was measured on a Hamamatsu FDSS μ-cell reader (Hamamatsu, Bridgewater, N.J.) and data was collected using the FDSS software.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A macromolecule comprising a plurality of linked peptide nucleic acid (PNA) strands, wherein each of said strands is independently composed of a plurality of nucleobase subunits, and each PNA strand is covalently linked to at least one other PNA strand via an amino acid linker, wherein the amino acid linker is a biological amino acid linker or a N,N-dimethyl-lysine linker.

2. The macromolecule of claim 1, wherein the linked PNA strands form a linear arrangement, a nonlinear arrangement, or a branched arrangement.

3.-4. (canceled)

5. The macromolecule of claim 1, wherein at least one amino acid linker mediates the linkage of more than two PNA strands.

6. The macromolecule of claim 1, wherein the linked PNA strands each independently comprise from 2 to about 50 nucleobase subunits.

7. (canceled)

8. The macromolecule of claim 1, wherein all of the linked PNA strands are of uniform length.

9. The macromolecule of claim 1, wherein at least one of the linked PNA strands differs in length from at least one other PNA strand.

10. The macromolecule of claim 1, wherein at least some of the linked PNA strands are individually bound to at least one complementary PNA strand to form a double stranded PNA segment.

11. The macromolecule of claim 10, wherein the ratio of linked PNA strands to complementary PNA strands is
from about 2:1 to about 10:1,

12.-13. (canceled)

14. The macromolecule of claim 10, wherein at least one nucleobase subunit of the complementary PNA comprises one or more gamma substituents.

15. (canceled)

16. The macromolecule of claim 14, wherein said gamma substituent is capable of binding to a protein on the surface of a cell, wherein the protein is a transmembrane protein, lipid-anchored protein, peripheral protein, cellular receptor, adhesion molecule, an integrin, a cadherin, a selectin, an addressin, a G protein-coupled receptor, or a toll-like receptor.

17.-19. (canceled)

20. The macromolecule of claim 14, wherein said gamma substituents, independently, are
—R—NX¹X², where:
R is a C₁-C₁₂ alkyl,
X^{l} and X² are independently selected from H biotin, fluorescein, thiazole orange, acridine, pyrene, Alexafluor Dyes, polypeptide, mannose, lactose, nucleic acid derivatives, oligonucleotides, RGD (Arg-Gly-Asp) cyclic RGD, cyclodextrins, porphyrins, polyhedral cage compounds containing boron, biotin, DOTA, DTPA, a crown ether, a cryptand, a pyridine-containing ligand, and calixarenes;
wherein at least one of X^{l} and X² are other than H.

21.-22. (canceled)

23. A pharmaceutical composition comprising a macromolecule of claim 1 and a pharmaceutically acceptable carrier.

24. A method of treating or inhibiting a disease state in a mammal comprising administering to said mammal a therapeutically effective amount of a composition of any one of the previous claims wherein at least some of the gamma substituents are selected to bind to a protein on the surface of a cell, wherein the protein is a transmembrane protein, lipid-anchored protein, peripheral protein, cellular receptor, or adhesion molecule.

25.-28. (canceled)

29. The method claim 24, wherein said disease state is related to, independently, cancer, HIV, diabetes (type 2), Chagas disease, chronic inflammatory diseases, and autoimmune diseases, anthrax or cholera.

30. (canceled)

31. A method of reducing metastasis of cancer cells in a mammal comprising administering to said mammal a therapeutically effective amount of a macromolecule of claim 14, wherein at least some of the gamma substituents are selected to bind to a protein on the surface of a cell, wherein the protein is a transmembrane protein, lipid-anchored protein, peripheral protein, cellular receptor, or adhesion molecule.

32.-35. (canceled)

36. The method of claim 24, wherein the administered composition comprises a gamma substituent of RGD (Arg-Gly-Asp) or cyclic RGD.

37. (canceled)

38. A method of forming a nanostructure platform comprising contacting a first PNA strand with a second PNA strand, wherein said first PNA strand comprises from 2 to 50 nucleobase subunits and is covalently linked to an amino acid linker; and wherein said second PNA strands comprise:
(i) from 2 to 50 nucleobase subunits; and
(ii) one or more gamma substituents;
wherein the ratio of said first PNA strand to said second PNA strand is at least 1:1 and said first PNA strands are complementary to a portion of said second PNA strands.

39.-40. (canceled)

41. The method of claim 38, wherein said gamma substituents, independently, are
—R—NX¹X², where:
R is a C₁-C₁₂ alkyl,
X^{l} and X² are independently selected from H, biotin, fluorescein, thiazole orange, acridine, pyrene, Alexafluor Dyes, polypeptide, mannose, lactose, nucleic acid derivatives, oligonucleotides, RGD (Arg-Gly-Asp) cyclic RGD, cyclodextrins, porphyrins, polyhedral cage compounds containing boron, biotin, DOTA, DTPA, a crown ether, a cryptand, a pyridine-containing ligand, and calixarenes;
wherein at least one of X^{l} and X² are other than H.

42.-43. (canceled)

44. A vaccine comprising a macromolecule of claim 14, wherein said gamma substituents comprise one or more of a bacterial or viral cell surface protein or an antigenic fragment thereof.

45. A method of detecting the presence of a cellular surface protein in a subject comprising administering to said subject a macromolecule of claim 1, wherein the compound is detectably labeled, wherein the detectable label is a fluorescent label, radiolabel, biotin, DOTA, DTPA, or a radionuclide.

46. (canceled)
